(19) United States
(12) Patent Application Publication
(10) Pub. No.: US 2024/0423638 A1
(43) Pub. Date: Dec. 26, 2024

(54) COMPRESSION DEVICE AND METHOD FOR DETACHING COMPRESSION DEVICE

(71) Applicant: TERUMO KABUSHIKI KAISHA, Shibuya-ku (JP)

(72) Inventor: Masakatsu KAWAURA, Hiratsuka city (JP)

(73) Assignee: TERUMO KABUSHIKI KAISHA, Shibuya-ku (JP)

(21) Appl. No.: 18/824,245

(22) Filed: Sep. 4, 2024

Related U.S. Application Data
(63) Continuation of application No. PCT/JP2023/007695, filed on Mar. 1, 2023.

(30) Foreign Application Priority Data
Mar. 23, 2022 (JP) 2022-047559

(51) Int. Cl. A61B 17/132; A61B 17/00; A61B 17/12; A61B 17/135

(52) U.S. Cl. CPC A61B 17/1325; A61B 17/135; A61B 2017/00539; A61B 2017/00557; A61B 2017/00951; A61B 2017/12004

(57) ABSTRACT

A compression device according to the present disclosure is provided with an adhesive sheet provided with an adhesive surface that can be adhered to a living body surface, an expansion member including an expandable expansion portion, a base member fixed to a fixing surface on a side opposite to the adhesive surface of the adhesive sheet and capable of pressing the expansion portion in an expanded state toward the living body surface, and a liquid guide member into which a release liquid can permeate, the liquid guide member provided with a main body adhered in the adhesive surface of the adhesive sheet, and the main body extending to a position of an outer edge of the adhesive surface.

### CROSS-REFERENCES TO RELATED APPLICATIONS

This application is a continuation of International Application No. PCT/JP2023/007695 filed on Mar. 1, 2023, which claims priority to Japanese Application No. 2022-047559 filed on Mar. 23, 2022, the entire content of both of which is incorporated herein by reference.

### TECHNOLOGICAL FIELD

The present disclosure generally relates to a compression device and a method for detaching the compression device.

### BACKGROUND DISCUSSION

Recently, in medical institutions, examinations or treatments of various modes are performed using a catheter. The catheter is percutaneously inserted into a blood vessel from a puncture site formed on the wrist, groin or the like, and is carried to a site to be examined or treated through the blood vessel. After completion of the examinations or treatment by a medical worker, a puncture needle, the catheter, and an elongated insertion member such as a sheath used for introducing the catheter into a living body are removed from the puncture site, and hemostasis of the puncture site is performed.

International Patent Application No. WO 2019/221201 discloses a compression device provided with an adhesive sheet and a compression member attached to the adhesive sheet capable of compressing a living body surface.

The compression device disclosed in International Patent Application No. WO 2019/221201 still has a room for improvement from the viewpoint of improving detachability from the living body surface after hemostasis of the puncture site.

### SUMMARY

A compression device is disclosed that can easily detach from a living body surface, and a method for detaching the compression device.

A compression device as a first aspect of the present disclosure is provided with an adhesive sheet provided with an adhesive surface that can be adhered to a living body surface, an expansion member including an expandable expansion portion, a base member fixed to a fixing surface on a side opposite to the adhesive surface of the adhesive sheet and capable of pressing the expansion portion in an expanded state toward the living body surface, and a liquid guide member into which a release liquid can permeate, the liquid guide member provided with a main body adhered in the adhesive surface of the adhesive sheet, and the main body extending to a position of an outer edge of the adhesive surface.

As one embodiment of the present disclosure, the adhesive sheet is not arranged at a position overlapping the expansion portion but is located around the expansion portion in plan view as seen in a thickness direction of the adhesive sheet, and the main body of the liquid guide member does not extend to a position of an inner edge of the adhesive surface but terminates in the adhesive surface.

As one embodiment of the present disclosure, the adhesive sheet is provided with a fixing portion to which the base member is fixed, and a non-fixing portion located outside the fixing portion in plan view as seen in a thickness direction of the adhesive sheet to which the base member is not fixed, the adhesive surface is provided with a first adhesive portion provided at a position of the fixing portion, and a second adhesive portion including the outer edge of the adhesive surface and is provided at a position of the non-fixing portion, and the main body of the liquid guide member is adhered only to the second adhesive portion.

As one embodiment of the present disclosure, the liquid guide member is provided with an outer extended portion continuous to the main body and extending outward from the outer edge of the adhesive surface.

As one embodiment of the present disclosure, the outer extended portion is folded back outside the outer edge of the adhesive surface and is fixed to the fixing surface.

As one embodiment of the present disclosure, in a case where a direction from the adhesive surface toward the fixing surface in a thickness direction of the adhesive sheet is defined as an upward direction, at least a part of the outer extended portion is configured to be able to protrude in the upward direction from the fixing surface.

A compression device in accordance with another aspect of the present disclosure includes: an adhesive sheet provided with an adhesive surface configured to be adhered to a living body surface; an expansion member including an expandable expansion portion; a base member fixed to a fixing surface on a side opposite to the adhesive surface of the adhesive sheet and configured to press the expansion portion in an expanded state toward the living body surface; and a liquid guide member into which a release liquid is configured to permeate, the liquid guide member provided with a main body adhered in the adhesive surface of the adhesive sheet, and wherein the main body of the guide member extends outward from an outer edge of the adhesive surface.

A method for detaching a compression device as a second aspect of the present disclosure is a method for detaching a compression device adhered to a living body surface, the compression device provided with an adhesive sheet provided with an adhesive surface that can be adhered to the living body surface, and a liquid guide member adhered in the adhesive surface into which a release liquid can permeate, the method provided with an impregnating step of impregnating the liquid guide member with the release liquid by allowing the release liquid to permeate from a liquid introduction portion of the release liquid provided at a position of an outer edge of the adhesive surface or a position outside the outer edge of the adhesive surface in the liquid guide member, and a detaching step of detaching the compression device from the living body surface by peeling the adhesive surface of the adhesive sheet off from the living body surface after the liquid guide member is impregnated with the release liquid.

According to the present disclosure, it is possible to provide a compression device easily detached from a living body surface, and a method for detaching the compression device.

### BRIEF DESCRIPTION OF THE DRAWINGS

FIG. 1 is a perspective view of a compression device as a first embodiment of the present disclosure.

FIG. 2 is a top view of the compression device illustrated in FIG. 1.

FIG. 3 is a bottom view of the compression device illustrated in FIG. 1.

FIG. 4 is a side view of the compression device illustrated in FIG. 1.

FIG. 5 is a cross-sectional view of the compression device taken along line I-I in FIGS. 2 and 3.

FIG. 6 is a side view as seen in the same direction as FIG. 4 in a case where an expansion portion of the compression device illustrated in FIG. 1 is in an expanded state.

FIG. 7 is a cross-sectional view at the same position as FIG. 5 in a case where the expansion portion of the compression device illustrated in FIG. 1 is in the expanded state.

FIG. 8 is a cross-sectional view of the compression device taken along line II-II in FIGS. 2 and 3.

FIG. 9 is a cross-sectional view of the compression device taken along line III-Ill in FIGS. 2 and 3.

FIG. 10 is a flowchart illustrating an example of a living body compression method performed using the compression device illustrated in FIG. 1.

FIG. 11A is a view illustrating an outline of an adhering step in FIG. 10.

FIG. 11B is a view illustrating an outline of an adhering step in FIG. 10.

FIG. 11C is a view illustrating an outline of a first compressing step in FIG. 10.

FIG. 11D is a view illustrating an outline of a removing step in FIG. 10.

FIG. 11E is a view illustrating an outline of a second compressing step in FIG. 10.

FIG. 11F is a view illustrating an outline of an impregnating step in FIG. 10.

FIG. 11G is a view illustrating an outline of a detaching step in FIG. 10.

FIG. 12 is a bottom view of a compression device as a second embodiment of the present disclosure.

FIG. 13 is a bottom view of a compression device as a third embodiment of the present disclosure.

FIG. 14 is a bottom view of a compression device as a fourth embodiment of the present disclosure.

FIG. 15 is a bottom view of a compression device as a variation of the compression device illustrated in FIG. 14.

FIG. 16 is a bottom view of a compression device as another variation of the compression device illustrated in FIG. 14.

FIG. 17 is a bottom view of a compression device as a fifth embodiment of the present disclosure.

FIG. 18 is a cross-sectional view of the compression device taken along line IV-IV in FIG. 17.

FIG. 19 is a cross-sectional view at the same position as FIG. 18, the view illustrating a state in which a main body of a liquid guide member is peeled off from an adhesive surface.

FIG. 20 is a cross-sectional view in the vicinity of a liquid guide member of a compression device as a sixth embodiment of the present disclosure.

FIG. 21A is a view illustrating a state in which insertion members for medical use are inserted from a living body surface into the femoral vein through connective tissue.

FIG. 21B is a view illustrating a state after removal of the insertion members for medical use from the state illustrated in FIG. 21A.

FIG. 22 is a view illustrating a state in which a perforation illustrated in FIG. 21B is narrowed or occluded by the compression device illustrated in FIG. 1.

FIG. 23 is a front view of the state illustrated in FIG. 22 as seen from a living body surface side.

### DETAILED DESCRIPTION

Hereinafter, embodiments of a compression device and a method for detaching the compression device according to the present disclosure is described by way of example with reference to the drawings. In the drawings, the same components are denoted by the same reference numerals.

### First Embodiment

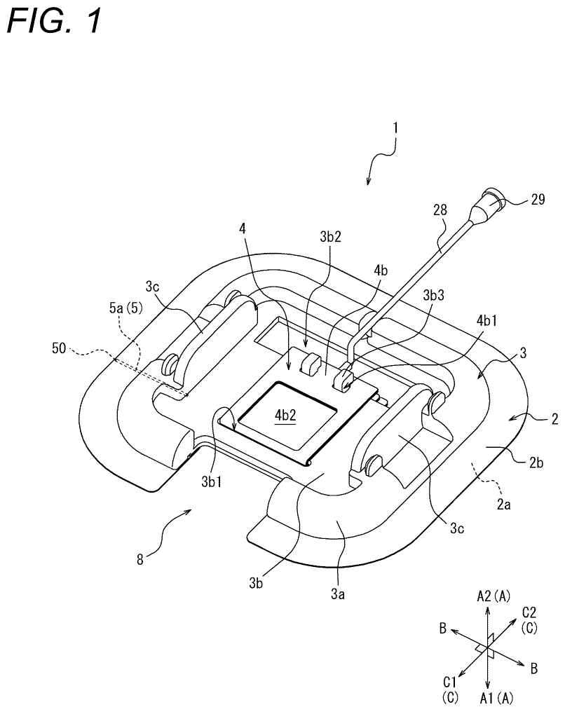

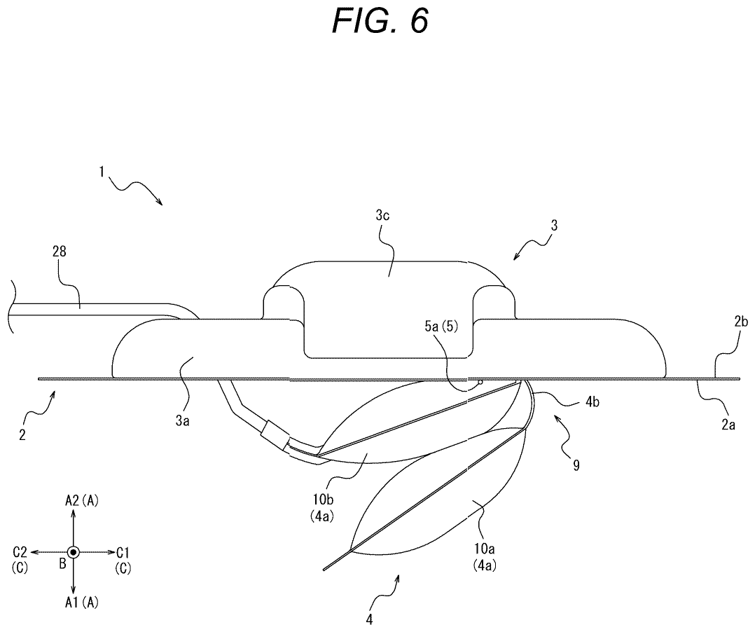

FIG. 1 is a perspective view of a compression device 1 as one embodiment of the present disclosure. As illustrated in FIG. 1, the compression device 1 is provided with an adhesive sheet 2, a base member 3, an expansion member 4, and a liquid guide member 5. The adhesive sheet 2 is provided with an adhesive surface 2a that can be adhered to a living body surface. The expansion member 4 includes an expandable expansion portion 4a (refer to FIG. 6 and the like). The base member 3 is fixed to a fixing surface 2b on a side opposite to the adhesive surface 2a of the adhesive sheet 2. In a state in which the adhesive surface 2a of the adhesive sheet 2 is adhered to the living body surface, the base member 3 presses the expansion portion 4a in an expanded state toward the living body surface. As a result, the expansion portion 4a of the expansion member 4 can compress the living body surface.

Hereinafter, for convenience of description, a thickness direction of the adhesive sheet 2 is simply referred to as a “thickness direction A”. In the thickness direction A, a direction from the fixing surface 2b toward the adhesive surface 2a is an adhering direction in which the compression device 1 is moved when the compression device 1 is adhered to the living body surface, and this is hereinafter sometimes simply referred to as a “downward direction A1” or “below” for convenience of description. Moreover, in the thickness direction A, a direction from the adhesive surface 2a toward the fixing surface 2b is a separating direction in which the compression device 1 is separated from the living body surface when the compression device 1 is peeled off from the living body surface, and this is hereinafter sometimes simply referred to as an “upward direction A2” or “above” for convenience of description.

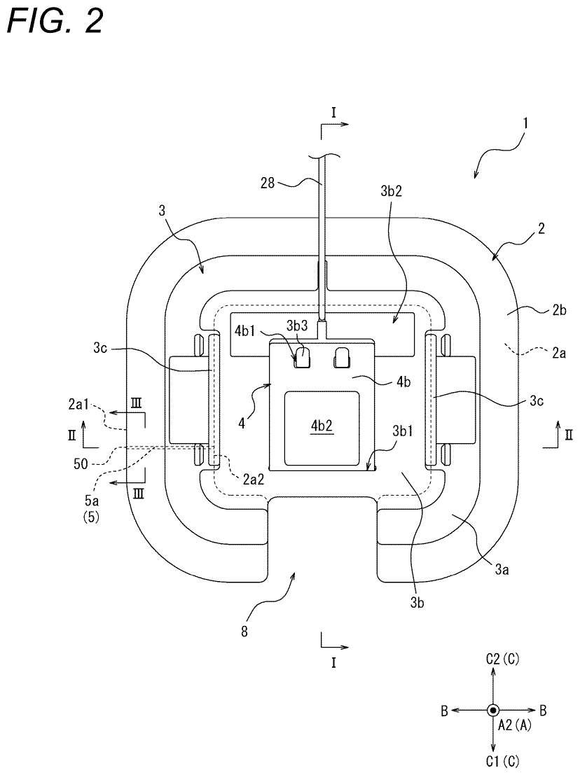

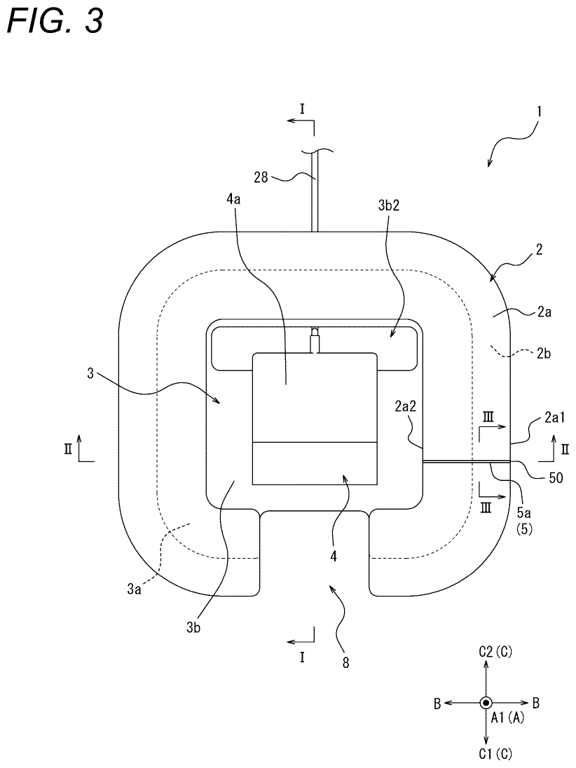

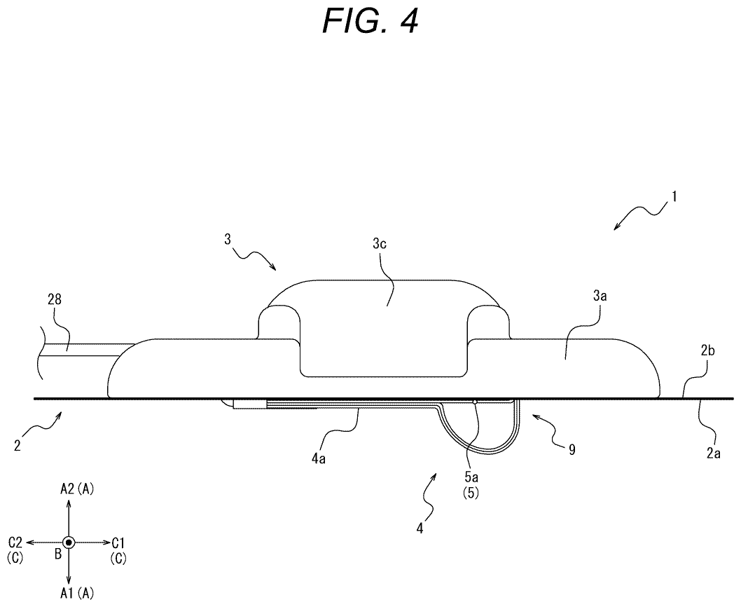

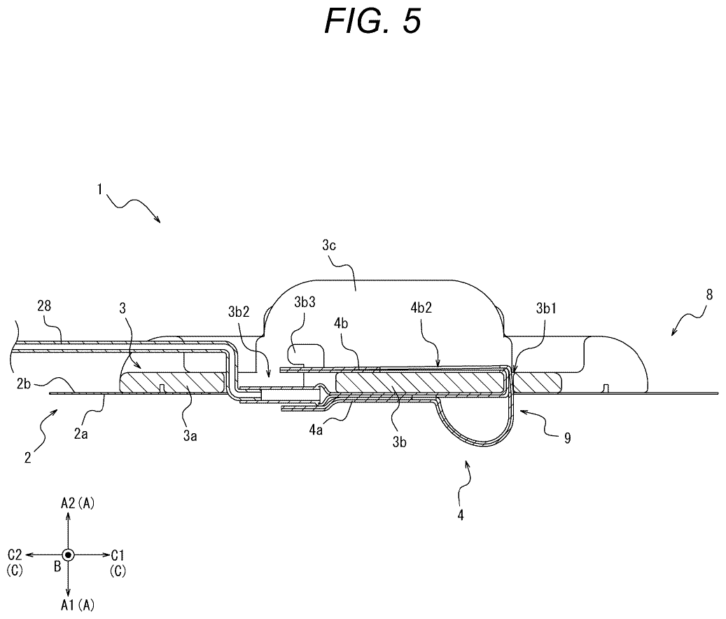

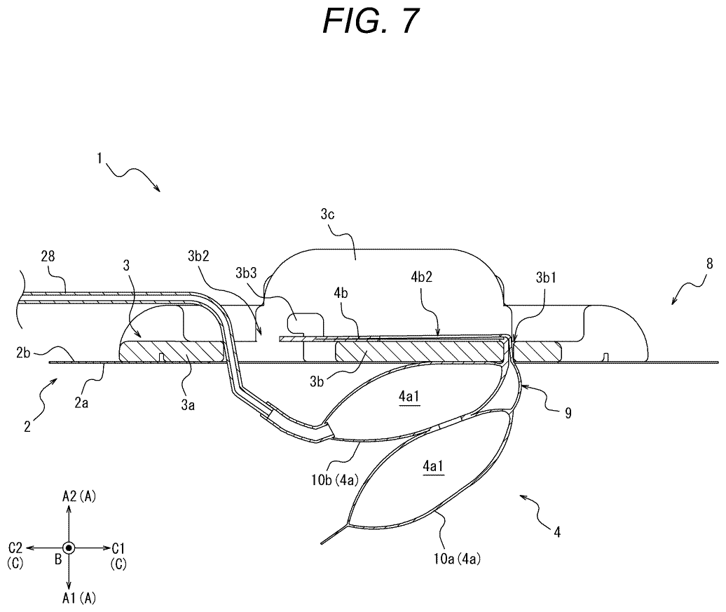

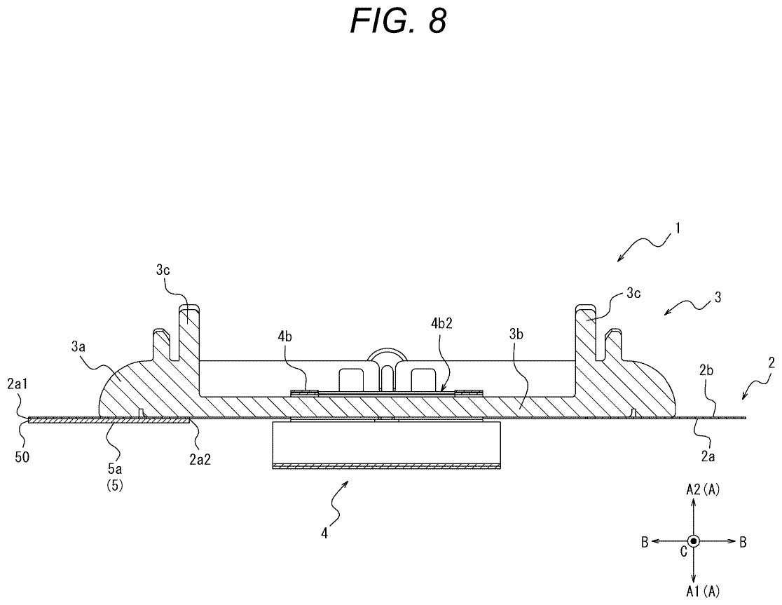

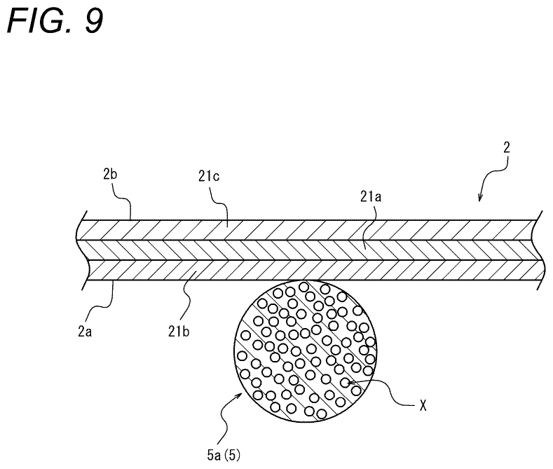

FIG. 2 is a top view of the compression device 1. FIG. 3 is a bottom view of the compression device 1. FIG. 4 is a side view of the compression device 1. FIG. 5 is a cross-sectional view taken along line I-I in FIGS. 2 and 3. FIGS. 1 to 5 illustrate a case where the expansion portion 4a of the compression device 1 is in a contracted state. In contrast, FIG. 6 is a side view as seen in the same direction as FIG. 4 in a case where the expansion portion 4a of the compression device 1 is in the expanded state. FIG. 7 is a cross-sectional view at the same position as FIG. 5 in a case where the expansion portion 4a of the compression device 1 is in the expanded state. FIG. 8 is a cross-sectional view taken along line II-II in FIGS. 2 and 3. FIG. 9 is a cross-sectional view taken along line III-III in FIGS. 2 and 3.

Hereinafter, out of plan views (refer to FIGS. 2 and 3) of the compression device 1 as seen in the thickness direction A, the plan view (refer to FIG. 2) as seen from above in the downward direction A1 is simply referred to as a “top view” for convenience of description. Out of plan views (refer to FIGS. 2 and 3) of the compression device 1 as seen in the thickness direction A, the plan view (refer to FIG. 3) as seen from below in the upward direction A2 is simply referred to as a “bottom view” for convenience of description. In a case where the top view and the bottom view are not particularly distinguished, they are simply referred to as the “plan view”. Unless otherwise specified, the simple terms of “plan view”, “top view”, and “bottom view” refer to the plan view, top view, and bottom view in a case where the expansion portion 4a of the expansion member 4 is in the contracted state.

As illustrated in FIGS. 1 to 9, the adhesive sheet 2 is provided with the adhesive surface 2a that can be adhered to the living body surface on a lower surface on one side in the thickness direction A. In the adhesive sheet 2, an upper surface on the other side in the thickness direction A forms the fixing surface 2b to which the base member 3 is fixed. The adhesive surface 2a of the adhesive sheet 2 is covered with a liner such as a release sheet in a state before use before the adhesive sheet 2 is adhered to the living body surface. The liner is peeled off from the adhesive surface 2a to be removed immediately before the adhesive surface 2a is adhered to the living body surface. The adhesive sheet 2 illustrated in FIGS. 1 to 9 illustrates a use state in which the liner is removed and the adhesive surface 2a is exposed.

As illustrated in FIGS. 6 and 7, the expansion member 4 is provided with the expandable expansion portion 4a. As described above, the base member 3 can press the expansion portion 4a in the expanded state toward the living body surface. In other words, the base member 3 presses the expansion portion 4a in the expanded state in the downward direction A1. Although it is described later in detail, the base member 3 of the present embodiment presses the expansion portion 4a toward the living body surface by interposing the expansion portion 4a in the expanded state between the same and the living body surface.

In this manner, the compression device 1 can compress a predetermined site on the living body surface by the base member 3 and the expansion member 4 in a state in which the adhesive surface 2a of the adhesive sheet 2 is adhered to the living body surface. Examples of the predetermined site on the living body surface include, for example, a wound and the vicinity of the wound on the living body surface formed when inserting an insertion member for medical use such as a puncture needle, a catheter, or a sheath into a blood vessel of a living body. After the insertion member for medical use described above is removed out of the living body, hemostasis can be achieved by compressing the wound or the vicinity of the wound on the living body surface with the compression device 1 for a predetermined time.

The compression device 1 is provided with the liquid guide member 5 adhered to the adhesive surface 2a of the adhesive sheet 2. The liquid guide member 5 is configured such that a release liquid can permeate into the liquid guide member 5. Examples of the release liquid include, but are not particularly limited to, a nonalcoholic silicone-based release agent, for example. The release liquid may be, for example, other hydrophobic release agents such as alcohols such as ethanol and isopropyl alcohol. The release liquid may contain, for example, a component that softens an adhesive forming the adhesive surface 2a.

The liquid guide member 5 is configured such that a release liquid can permeate into the liquid guide member 5. The permeation of the release liquid into the liquid guide member 5 may be achieved by, for example, a capillary phenomenon, use of the liquid guide member 5 having high wettability, use of the release liquid having low surface tension or the like. As illustrated in FIG. 9, the liquid guide member 5 defines a large number of voids X into which the release liquid can permeate. Examples of the liquid guide member 5 include, for example, a nonwoven fabric, a woven fabric, a knitted fabric, a twisted yarn, a porous body such as a foam and the like. In a case where the liquid guide member 5 is formed of an assembly of fibers such as a nonwoven fabric, a woven fabric, a knitted fabric, and a twisted yarn, constituent fibers are more preferably hollow fibers.

The liquid guide member 5 is provided with a main body 5a adhered in the adhesive surface 2a of the adhesive sheet 2. The main body 5a extends to a position of an outer edge 2a1 of the adhesive surface 2a. The position of the outer edge 2a1 of the adhesive surface 2a of the present embodiment coincides with a position of an outer edge of the adhesive sheet 2. By providing the liquid guide member 5 provided with such main body 5a, when the compression device 1 is detached from the living body surface after hemostasis is completed, the release liquid can be allowed to permeate into the main body 5a of the liquid guide member 5 from the position of the outer edge 2a1 of the adhesive surface 2a in a state of being adhered to the living body surface. As a result, the release liquid can be supplied to a minute gap between the living body surface and the adhesive surface 2a via the main body 5a of the liquid guide member 5. Therefore, the adhesive surface 2a can be rather easily peeled off from the living body surface. That is, it is possible to implement the compression device 1 easily detached from the living body surface. Note that, in a state before use of the compression device 1, the liner such as the release sheet is adhered to the adhesive surface 2a to which the liquid guide member 5 is adhered so as to cover the liquid guide member 5, and protects the adhesive surface 2a and the liquid guide member 5.

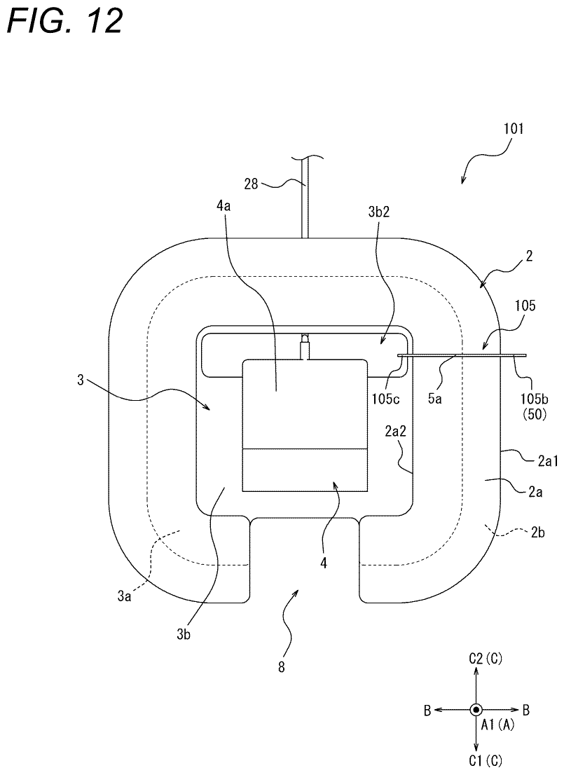

The liquid guide member 5 of the present embodiment is formed only of the main body 5a, but is not limited to this configuration. The liquid guide member 5 is preferably provided with an outer extended portion continuous to the main body 5a. The outer extended portion of the liquid guide member 5 is described later in detail (refer to FIGS. 12 to 20).

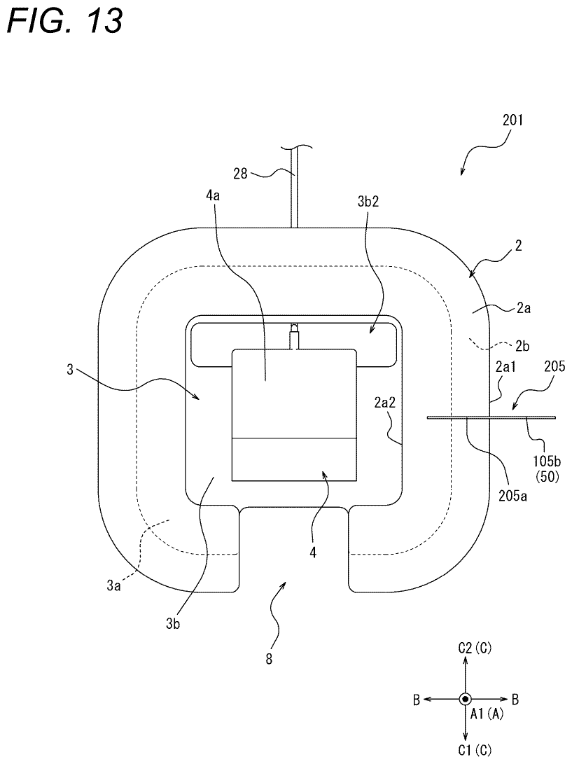

The main body 5a of the liquid guide member 5 of the present embodiment extends from the outer edge 2a1 to an inner edge 2a2 of the adhesive surface 2a of the adhesive sheet 2, but this is not limited to this configuration. The main body 5a does not extend from the outer edge 2a1 to the inner edge 2a2 of the adhesive surface 2a, and may terminate in the adhesive surface 2a. Such main body of the liquid guide member 5 is described later in detail (refer to FIGS. 13 to 20).

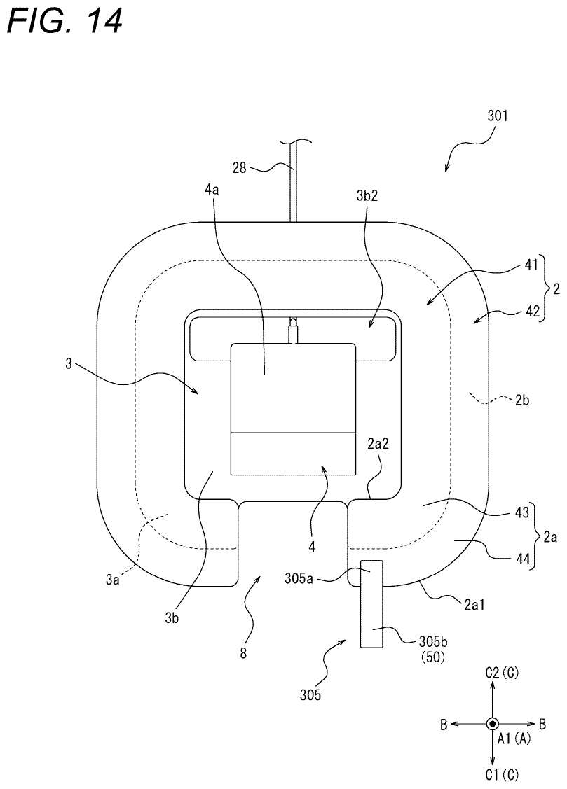

Moreover, the liquid guide member 5 of the present embodiment is a linear member of which cross section orthogonal to a longitudinal direction has a substantially circular outer shape (hereinafter, simply referred to as a “cross-sectional outer shape”), but is not limited to this configuration. The cross-sectional outer shape of the liquid guide member 5 may be, for example, an oval shape, a rectangular shape or the like, and is not especially limited. An example of another shape of the liquid guide member 5 is described later (refer to FIG. 14 and the like).

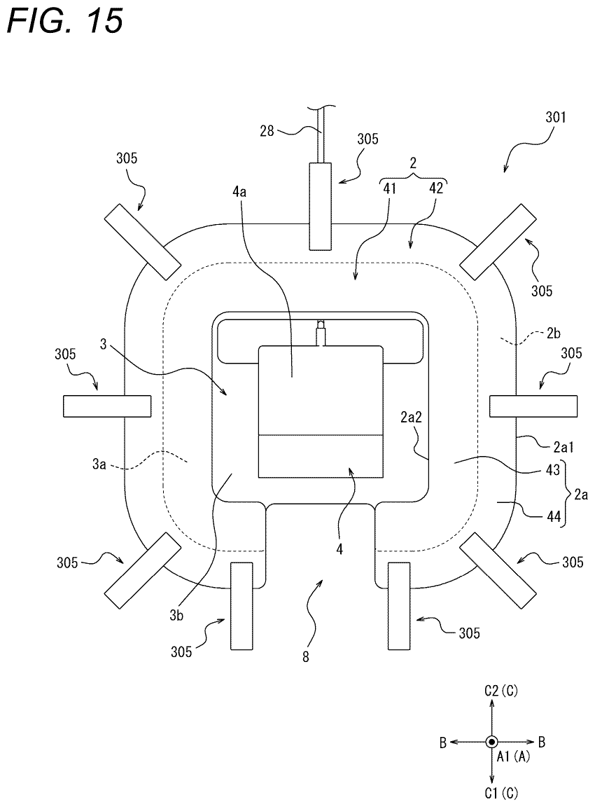

The compression device 1 of the present embodiment is provided with only one liquid guide member 5, but is not limited to this configuration. The compression device 1 may be provided with, for example, a plurality of liquid guide members 5. Such configuration is described later in detail (refer to FIG. 15).

Hereinafter, the compression device 1 of the present embodiment is described in further detail.

### Adhesive Sheet 2

The adhesive sheet 2 has flexibility. Therefore, the adhesive sheet 2 can be deformed along a shape of the living body surface. The adhesive surface 2a easily follows deformation of the living body surface. As a result, it is possible to suppress unintended peeling of the compression device 1 from the living body surface.

The adhesive surface 2a of the adhesive sheet 2 of the present embodiment is formed of an entire lower surface of the adhesive sheet 2. The adhesive surface 2a of the adhesive sheet 2 may be provided only in a partial region of the lower surface of the adhesive sheet 2.

As illustrated in FIG. 9, the adhesive sheet 2 of the present embodiment is formed of a plurality of layers. Specifically, the adhesive sheet 2 of the present embodiment is provided with a base layer 21a, an adhesive layer 21b, and a surface layer 21c.

The base layer 21a can be formed of, for example, a thin resin sheet. More specifically, the base layer may be formed of, for example, a white spun-lace nonwoven fabric of polyester fibers. A thickness thereof is in a range of 5 μm to 150 μm, for example, 30 μm. Note that, the material of the base layer 21a is not limited to polyester, and may be, for example, an acrylic polymer, polyethylene, an ethylene-vinyl acetate copolymer, polyurethane, a polyamide derivative or the like.

The adhesive layer 21b may be formed of, for example, an adhesive such as a rubber-based adhesive, an acrylic adhesive, or a silicon-based adhesive. The adhesive layer 21b is stacked on the base layer 21a directly or indirectly with another layer interposed between the adhesive layer 21b and the base layer 21a (in this embodiment, directly). The adhesive surface 2a, which is the lower surface of the adhesive sheet 2, of the present embodiment is formed of the adhesive layer 21b.

The surface layer 21c is stacked on the base layer 21a directly or indirectly (in this embodiment, directly) on a side opposite to the adhesive layer 21b with the base layer 21a interposed between the surface layer 21c and the adhesive layer 21b. The surface layer 21c can be formed of, for example, a resin sheet having a thickness of about 5 μm to 50 μm. More specifically, the surface layer 21c of the present embodiment is formed of a polyurethane sheet having a thickness of about 5 μm to 50 μm. Note that, the material of the surface layer 21c is not limited to polyurethane. As the material of the surface layer 21c, polyester, polyamide, polyamideimide, polyethylene, polypropylene, polycarbonate, polyvinyl chloride, a fluororesin or the like can be used. The fixing surface 2b, which is an upper surface of the adhesive sheet 2, of the present embodiment is formed of the surface layer 21c.

The adhesive sheet 2 may include another layer in addition to the base layer 21a, the adhesive layer 21b, and the surface layer 21c described above. The adhesive sheet 2 may be formed of the base layer 21a and the adhesive layer 21b. In such a case, the fixing surface 2b, which is the upper surface of the adhesive sheet 2, may be formed of the base layer 21a.

The adhesive sheet 2 of the present embodiment may have translucency in the thickness direction A. Herein, to “have translucency in the thickness direction A” means to be translucent or transparent in the thickness direction A so that it is possible to see through. By doing so, the living body surface located below the adhesive sheet 2 can be easily visually recognized from above the adhesive sheet 2 through the adhesive sheet 2.

Moreover, the adhesive sheet 2 may be a non-woven fabric tape having one surface provided with a sticky adhesive as an adhesive, for example. The adhesive sheet 2 may be a double-sided tape in which adhesive layers are provided on both sides of the base layer, for example. In a case where the adhesive sheet 2 is formed of the double-sided tape, the base member 3 can be fixed to the adhesive sheet 2 by adhering the base member 3 to one adhesive layer as the fixing surface 2b of the adhesive sheet 2.

The adhesive sheet 2 of the present embodiment has a substantially C-shaped outer shape in plan view as seen in the thickness direction A of the adhesive sheet 2. As illustrated in FIGS. 2 and 3, the adhesive sheet 2 of the present embodiment covers an entire lower surface of an outer edge portion of the base member 3 except for a part of the base member 3. A gap is defined between both ends of the adhesive sheet 2 extending in a substantially C-shape of the present embodiment. The gap forms a receiving portion 8 capable of receiving the insertion member for medical use in a state of being inserted or to be inserted into the blood vessel of the living body.

### Base Member 3

As described above, the base member 3 is fixed to the upper surface as the fixing surface 2b of the adhesive sheet 2. More specifically, the base member 3 of the present embodiment covers a central region surrounded by the adhesive sheet 2 extending in the substantially C-shape in plan view (refer to FIGS. 2 and 3) as seen in the thickness direction A. The outer edge portion of the base member 3 is arranged at a position overlapping the adhesive sheet 2 in the thickness direction A, and is fixed to the upper surface of the adhesive sheet 2. The base member 3 presses the expansion portion 4a of the expansion member 4 in the central region surrounded by the adhesive sheet 2 having the substantially C-shape in plan view. As a result, the expansion portion 4a of the expansion member 4 can compress the living body surface by being pressed by the base member 3 toward the living body surface.

More specifically, the base member 3 of the present embodiment is provided with a frame portion 3a, a cover portion 3b, and a base gripping portion 3c. The frame portion 3a extends in a substantially C-shape in plan view. The frame portion 3a is fixed to the upper surface of the adhesive sheet 2. The cover portion 3b is continuous to an inner side of the frame portion 3a in plan view. The cover portion 3b covers the central region surrounded by the adhesive sheet 2 having the substantially C-shape in plan view. The cover portion 3b is formed of a plate-shaped portion that is flat in the thickness direction A. The base gripping portion 3c protrudes from the cover portion 3b in the upward direction A2. The base gripping portion 3c of the present embodiment is formed of two gripping plate portions protruding from two opposing portions of the cover portion 3b in plan view in the upward direction A2. The two gripping plate portions as the base gripping portion 3c are arranged to be opposed to each other. A user such as a doctor who uses the compression device 1 can hold the compression device 1 by gripping so as to interpose the two gripping plate portions arranged so as to be opposed to each other.

Hereinafter, an opposing direction (horizontal direction in FIG. 2) in which the two gripping plate portions as the base gripping portion 3c are opposed to each other in plan view is sometimes referred to as a “width direction B of the compression device 1” or simply as a “width direction B”. A direction (vertical direction in FIG. 2) orthogonal to the width direction B in plan view is sometimes referred to as a “front-rear direction C of the compression device 1” or simply as a “front-rear direction C”. Moreover, an end of the base member 3 located on the receiving portion 8 side in plan view is defined as a front end (a lower end in FIG. 2) of the base member 3, and an end of the base member 3 located on a side opposite to the receiving portion 8 side in plan view is defined as a rear end (an upper end in FIG. 2) of the base member 3. That is, in the present embodiment, a direction from the rear end to the front end of the base member 3 in plan view in the front-rear direction C is referred to as a “forward direction C1”. A direction from the front end to the rear end of the base member 3 in plan view in the front-rear direction C is referred to as a “rearward direction C2”.

The cover portion 3b interposes the expansion portion 4a in the expanded state between the same and the living body surface. Specifically, the cover portion 3b of the present embodiment is located above the expansion portion 4a. Therefore, when the expansion portion 4a expands in a state in which the adhesive surface 2a of the adhesive sheet 2 is adhered to the living body surface, the expansion portion 4a is interposed between the cover portion 3b and the living body surface from above and below.

The cover portion 3b of the present embodiment defines another through-hole 3b2 penetrating in the thickness direction A in addition to a through-hole 3b1 through which an extended portion 4b of the expansion member 4 to be described later is inserted. A tube 28 to be described later is inserted into the through-hole 3b2 of the present embodiment. The cover portion 3b is provided with a locking protrusion 3b3 protruding in the upward direction A2.

Examples of the material of the base member 3 according to the present embodiment can include a resin material, for example. Examples of the resin material can include, for example, thermoplastic resins used in injection molding such as an ABS resin, an AS resin, polyethylene, polypropylene, polystyrene, polyvinyl chloride, a polyvinylidene chloride resin, a polyphenylene oxide, thermoplastic polyurethane, polymethylene methacrylate, polyoxyethylene, a fluororesin, polycarbonate, polyamide, an acetal resin, an acrylic resin, and polyethylene terephthalate, thermosetting resins such as a phenol resin, an epoxy resin, a silicone resin, and unsaturated polyester and the like.

The base member 3 of the present embodiment has translucency in the thickness direction A. By doing so, the position of the expansion portion 4a of the expansion member 4 located below the base member 3 can be confirmed from above the base member 3 through the cover portion 3b of the base member 3.

### Expansion Member 4

The expansion member 4 of the present embodiment is provided with the expansion portion 4a and the extended portion 4b.

The expansion portion 4a can press the living body surface in a state in which the adhesive surface 2a is adhered to the living body surface (hereinafter, simply referred to as an “adhered state”). Specifically, the expansion portion 4a of the present embodiment can press the living body surface by extending in the thickness direction A as a direction orthogonal to the adhesive surface 2a in the adhered state. The expansion portion 4a of the present embodiment can press the living body surface at a position at which there is no adhesive sheet 2 in plan view. That is, the expansion portion 4a of the present embodiment can press the living body surface without an intervention of the adhesive sheet 2.

More specifically, the expansion member 4 of the present embodiment can press the living body surface in the central region surrounded by the adhesive sheet 2 having the substantially C-shape in plan view. The expansion portion 4a of the present embodiment is located below the base member 3 on one side in the thickness direction A. The expansion portion 4a is arranged between the living body surface and the cover portion 3b of the base member 3 in the adhered state. The expansion portion 4a can be expanded in the thickness direction A by supply of a fluid in the adhered state. When the expansion portion 4a expands in the adhered state, the expansion portion 4a receives a reaction force from the cover portion 3b of the base member 3 to press the living body surface. In the compression device 1 of the present embodiment, in a case where the expansion portion 4a is in the contracted state in the adhered state (refer to FIGS. 1 to 5), the expansion portion 4a does not compress the living body surface. The term “does not compress” as used herein means not only a state of not compressing at all, but also a state of compressing with an extremely weak force to such an extent that a hemostatic effect cannot be obtained by contacting the living body surface. In contrast, in the compression device 1 of the present embodiment, in a case where the expansion portion 4a is in the expanded state in the adhered state (refer to FIGS. 6 and 7), the expansion portion 4a compresses the living body surface.

As illustrated in FIG. 7, the expansion portion 4a of the present embodiment defines a housing space 4a1 capable of housing a fluid such as gas in the housing space 4a1. The expansion portion 4a of the present embodiment is provided with two balloon portions 10a and 10b overlapping in the thickness direction A in the contracted state. The two balloon portions 10a and 10b of the present embodiment are connected in such a manner that the insides of the two balloon portions 10a and 10b communicate with each other. The housing space 4a1 of the present embodiment is formed of an inner space in which the two balloon portions 10a and 10b communicate with each other. Note that, the expansion portion 4a may be provided with only one balloon portion. As in the present embodiment, in a case where the expansion portion 4a is provided with a plurality of balloon portions, the plurality of balloon portions may define separate housing spaces that do not communicate with each other.

As illustrated in FIG. 7, the expansion portion 4a is expandable in the downward direction A1 by supply of the fluid to the housing space 4a1. The expansion portion 4a of the present embodiment expands in the downward direction A1 by changing the state from the above-described contracted state (refer to FIGS. 1 to 5 and the like) to the expanded state (refer to FIGS. 6 and 7), and takes a posture capable of compressing the living body surface. More specifically, when the fluid is supplied to the housing space 4a1, the expansion portion 4a receives a reaction force from the lower surface of the cover portion 3b of the base member 3 and expands in the downward direction A1. The fluid supplied to the housing space 4a1 of the expansion portion 4a is not limited to a gas and may be a liquid.

As illustrated in FIGS. 4 and 5, the expansion portion 4a in the contracted state is arranged along the lower surface of the cover portion 3b of the base member 3. The housing space 4a1 of the expansion portion 4a communicates with the tube 28 extending to the outside of the base member 3. A fluid is supplied to the housing space 4a1 of the expansion portion 4a from a fluid supply instrument such as a syringe connected to an inflation port as a connection portion 29 provided at an end of the tube 28 through the tube 28. As a result, the state of the expansion portion 4a can be changed between the contracted state (refer to FIGS. 1 to 5) and the expanded state (refer to FIGS. 6 and 7).

The extended portion 4b can be a tab-shaped portion extending in a sheet shape from the expansion portion 4a. The extended portion 4b has flexibility. The extended portion 4b is wound around the cover portion 3b of the base member 3. As a result, the extended portion 4b extends from the expansion portion 4a to the upper surface side of the cover portion 3b on the side opposite to the expansion portion 4a with the cover portion 3b interposed between the extended portion 4b and the expansion portion 4a. The extended portion 4b is locked to the cover portion 3b on the upper surface side of the cover portion 3b.

As described above, the cover portion 3b of the present embodiment defines the through-hole 3b1 penetrating in the thickness direction A. The cover portion 3b of the present embodiment is provided with a locking protrusion 3b3 protruding in the upward direction A2. The extended portion 4b of the present embodiment is wound around the cover portion 3b through the through-hole 3b1. More specifically, the extended portion 4b of the present embodiment extends from below in which the expansion portion 4a is located to the above on the side opposite to the same with the cover portion 3b interposed between the extended portion 4b and the expansion portion 4a through the through-hole 3b1. The extended portion 4b of the present embodiment is wound around the cover portion 3b along the inner surface defining the through-hole 3b1 of the cover portion 3b and the upper surface of the cover portion 3b. A locking hole 4b1 into which the locking protrusion 3b3 is fitted is formed in the extended portion 4b on the upper surface side of the cover portion 3b. By fitting the locking protrusion 3b3 into the locking hole 4b1, the extended portion 4b is positioned on the cover portion 3b. The extended portion 4b is wound from the lower surface side to the upper surface side of the cover portion 3b at a position on the receiving portion 8 side with respect to the expansion portion 4a. That is, the through-hole 3b1 of the present embodiment is located on the receiving portion 8 side with respect to the expansion portion 4a. Therefore, the expansion portion 4a and the extended portion 4b of the expansion member 4 of the present embodiment are curved in a substantially U-shape as a whole in the cross-sectional view illustrated in FIG. 5. As a result, the expansion portion 4a can expand while rotating with a portion connected to the expansion portion 4a of the extended portion 4b as a hinge 9. In the side view illustrated in FIG. 4 and the cross-sectional view illustrated in FIG. 5, a part of the expansion portion 4a and the extended portion 4b of the present embodiment protrude downward by elasticity of the resin sheet forming the expansion portion 4a and the extended portion 4b. The above-described “substantially U-shape” means an entire shape of the expansion portion 4a and the extended portion 4b excluding a part of the expansion portion 4a and the extended portion 4b protruding downward by the elasticity of the resin sheet described above.

The expansion portion 4a can expand not only in the thickness direction A but also in a direction inclined with respect to the thickness direction A by rotating with the hinge 9 of the extended portion 4b. As described above, the expansion member 4 of the present embodiment is fixed to the cover portion 3b in a state in which the sheet-shaped extended portion 4b is wound over the upper and lower surfaces of the cover portion 3b through the through-hole 3b1 of the cover portion 3b of the base member 3. Therefore, when expanding, the expansion portion 4a expands while rotating around the rotation center with the hinge 9, which is a portion connected to the expansion portion 4a of the extended portion 4b below the through-hole 3b1, as the rotation center.

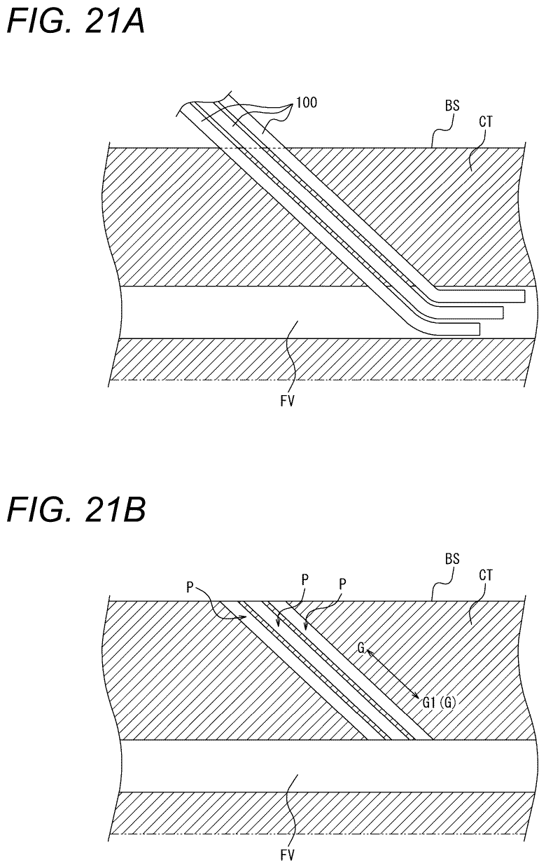

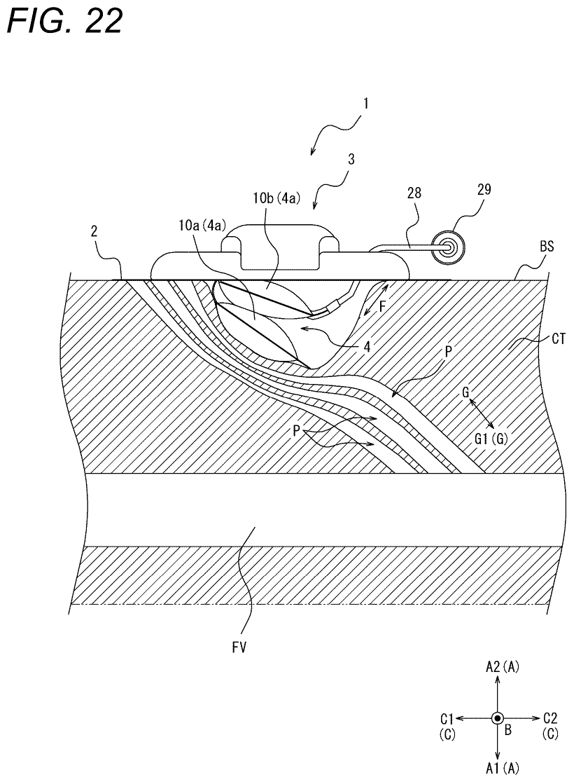

More specifically, the two balloon portions 10a and 10b of the expansion portion 4a of the present embodiment are arranged in a state overlapping in the thickness direction A in the contracted state. One end of each of the two balloon portions 10a and 10b is attached to the extended portion 4b. That is, one end side of the two balloon portions 10a and 10b is restrained by the extended portion 4b. Therefore, even when the two balloon portions 10a and 10b are expanded, a separation distance between the two balloon portions 10a and 10b is limited on the one end side. In contrast, the other end side of the two balloon portions 10a and 10b is not restrained at all. Therefore, when the two balloon portions 10a and 10b are expanded, the separation distance between the two balloon portions 10a and 10b is not limited on the other end side. That is, in the two balloon portions 10a and 10b of the expansion portion 4a of the present embodiment, the other end side not attached to the extended portion 4b rotates around the rotation center with one end side attached to the extended portion 4b as the rotation center. In this manner, the expansion portion 4a of the present embodiment expands in the direction inclined with respect to the thickness direction A. With the expansion portion 4a that expands in the direction inclined with respect to the thickness direction A, a perforation P (refer to FIG. 21B) to be described later is easily narrowed or blocked. The perforation P being narrowed or occluded by the compression device is described later in detail (refer to FIG. 22). Note that, a configuration for expanding in the direction inclined with respect to the thickness direction A is not limited to the configuration of the expansion member 4 of the present embodiment.

The expansion member 4 of the present embodiment is formed of a member having translucency, and it is possible to see through in the direction orthogonal to the adhesive surface 2a. As described above, the same applies to the cover portion 3b of the base member 3 of the present embodiment. Therefore, according to the compression device 1 of the present embodiment, a compression position on the living body surface can be visually recognized through the cover portion 3b of the base member 3 and the expansion portion 4a and the extended portion 4b of the expansion member 4. As in the present embodiment, the extended portion 4b may be provided with a visual recognition through-hole 4b2 in order to facilitate visual recognition of the compression position on the living body surface.

As a constituent material of the expansion portion 4a and the extended portion 4b of the expansion member 4, for example, soft polyvinyl chloride, polyurethane, polyethylene, polypropylene, polyester, an ethylene-vinyl acetate copolymer (EVA), silicone, or a flexible material obtained by mixing any of these materials can be used.

### Liquid Guide Member 5

After hemostasis by the compression device 1 is completed, the compression device 1 is detached from the living body surface. At that time, the adhesive surface 2a of the adhesive sheet 2 is peeled off from the living body surface. An adhering force of the adhesive surface 2a of the adhesive sheet 2 to the living body surface is required to be relatively high in order to maintain a compressing force on the living body surface by the expansion portion 4a. In contrast, the high adhering force of the adhesive surface 2a to the living body surface might give a large stimulation to the living body when this is peeled off after hemostasis by the compression device 1 is completed. There is a risk of damaging the skin at the time of peeling. In contrast, since the compression device 1 is provided with the liquid guide member 5, it is possible to reduce the stimulation applied to the living body and the damage of the skin when the adhesive surface 2a is peeled off from the living body surface.

The liquid guide member 5 of the present embodiment is described in detail with reference to FIGS. 1, 2, 3, 8, and 9. The liquid guide member 5 is provided with the main body 5a adhered in the adhesive surface 2a of the adhesive sheet 2 as described above. The main body 5a extends to a position of an outer edge 2a1 of the adhesive surface 2a. The position of the outer edge 2a1 of the adhesive surface 2a of the present embodiment coincides with the position of the outer edge of the adhesive sheet 2 as described above. Therefore, the release liquid can be allowed to permeate into the main body 5a of the liquid guide member 5 from the position of the outer edge of the adhesive sheet 2. That is, a portion at the position of the outer edge 2a1 of the adhesive surface 2a of the main body 5a forms a liquid introduction portion 50 of the release liquid. The release liquid permeates into the main body 5a from the liquid introduction portion 50. Moreover, the release liquid spreads so as to enter a minute gap between the adhesive surface 2a and the living body surface in the vicinity of the main body 5a via the main body 5a. In this manner, the adhering force of the adhesive surface 2a to the living body surface can be weakened. Therefore, when the adhesive surface 2a is peeled off from the living body surface, it is possible to reduce the stimulation applied to the living body and the damage of the skin. That is, it is possible to rather easily detach the compression device 1 after hemostasis is completed from the living body surface.

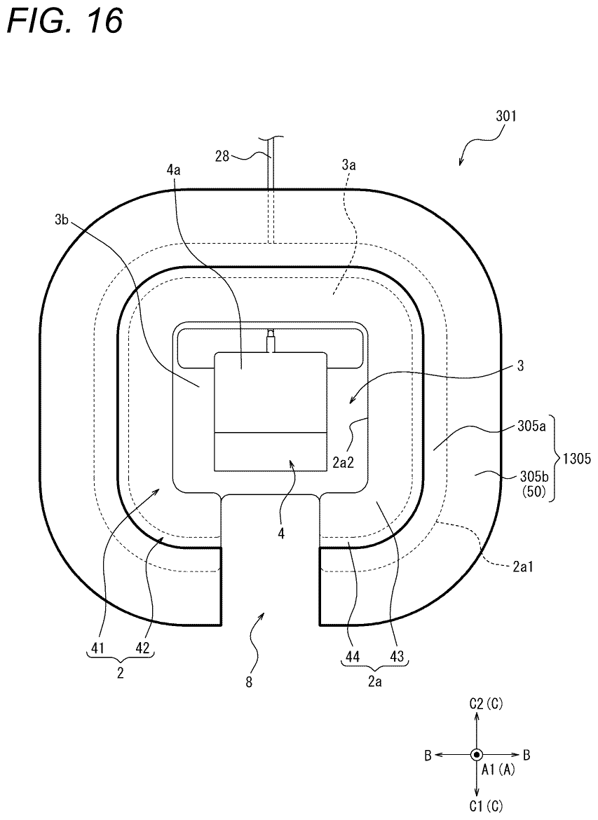

Although only one liquid guide member 5 of the present embodiment is arranged at a position on one side in the width direction B of the adhesive sheet 2 extending in the substantially C-shape in plan view (refer to FIGS. 2 and 3) as seen in the thickness direction A, it is not limited to this configuration. The position at which the liquid guide member 5 is provided may be, for example, a position on the other side in the width direction B. The position at which the liquid guide member 5 is provided may be, for example, a position on one side or the other side in the front-rear direction C. Moreover, a plurality of liquid guide members 5 may be arranged at optional positions as described later (refer to FIG. 15). Moreover, the liquid guide member 5 may be arranged along the outer edge of the adhesive sheet 2 over an entire outer edge of the adhesive sheet 2 as described later (refer to FIG. 16).

A constituent material of the liquid guide member 5 is not particularly limited. Examples of the constituent material of the liquid guide member 5 can include, for example, polypropylene, polyester, an acrylic polymer, polyethylene, an ethylene-vinyl acetate copolymer, polyurethane, a polyamide derivative, silk or the like.

### Living Body Compression Method Performed Using Compression Device 1

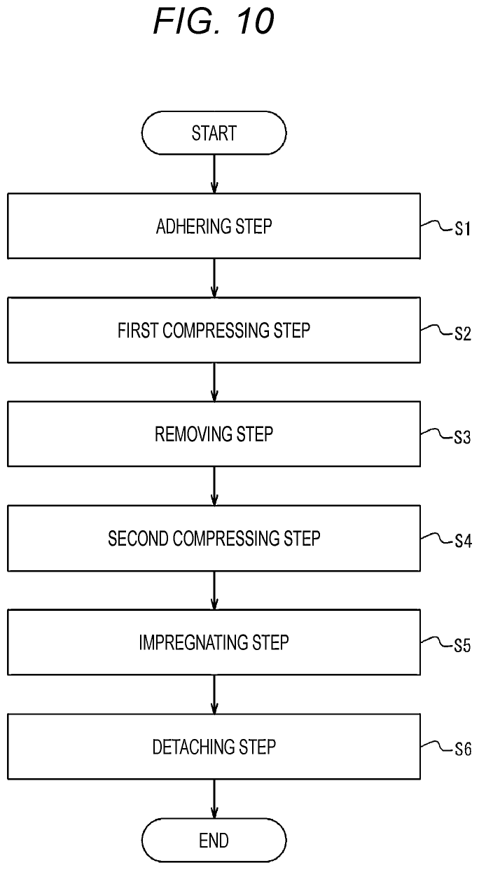

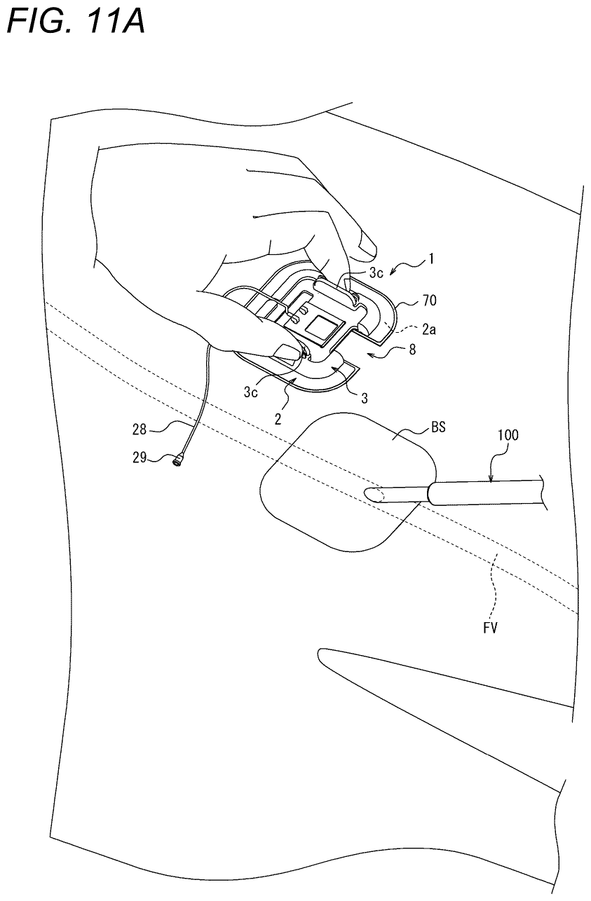

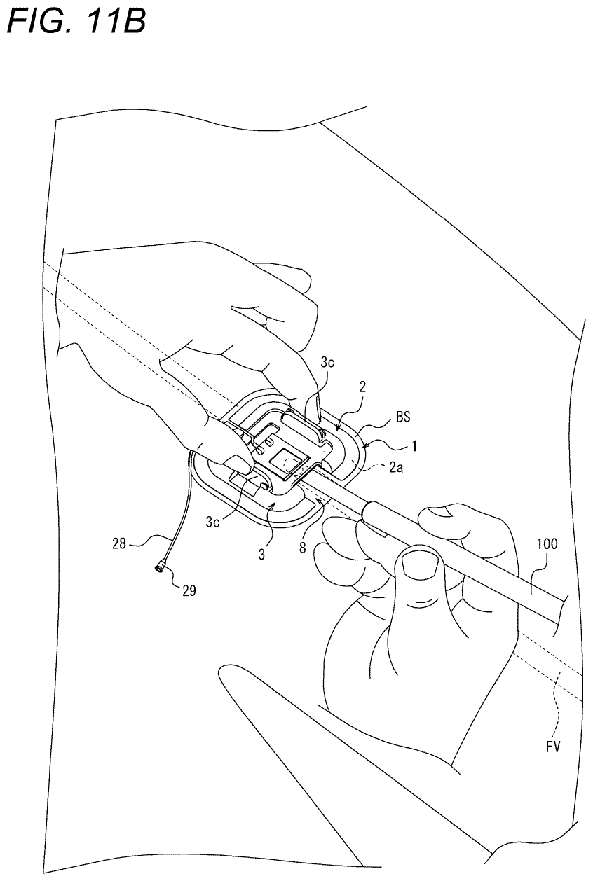

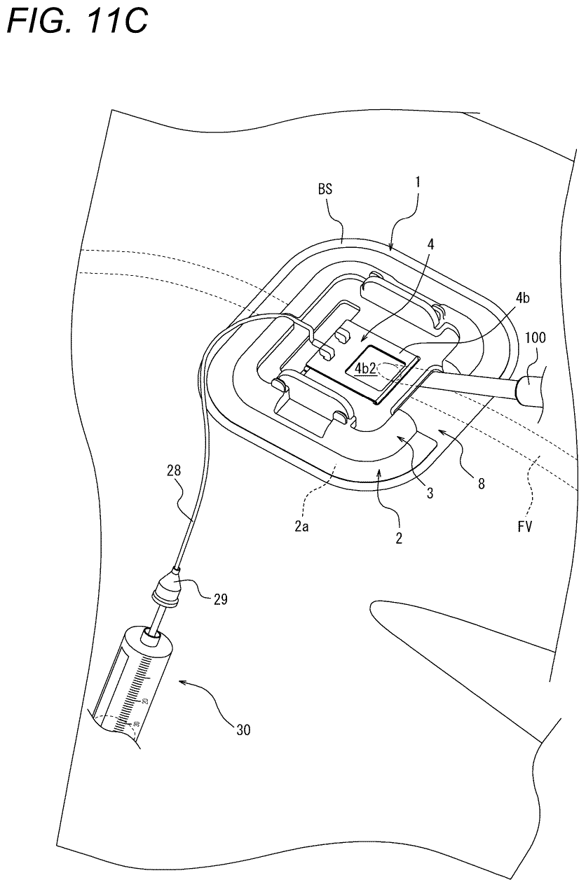

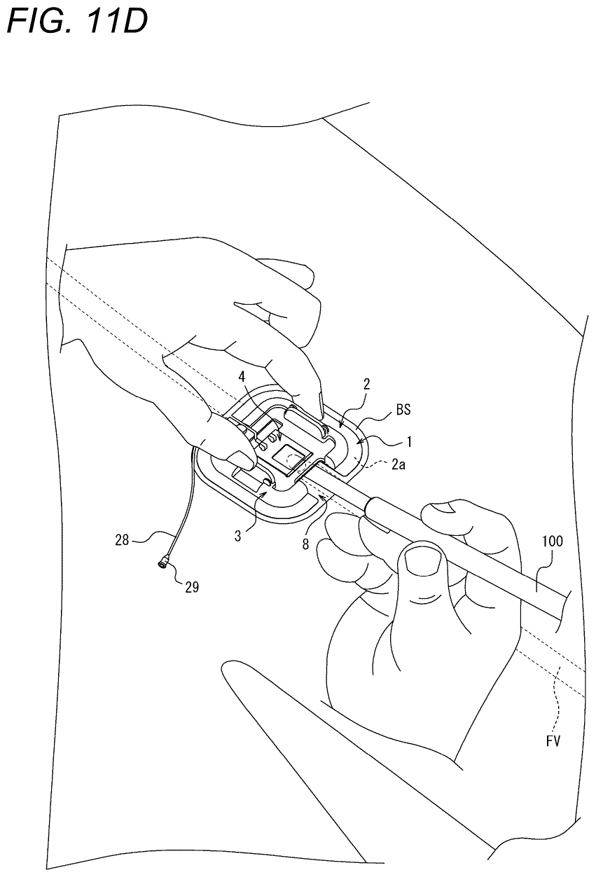

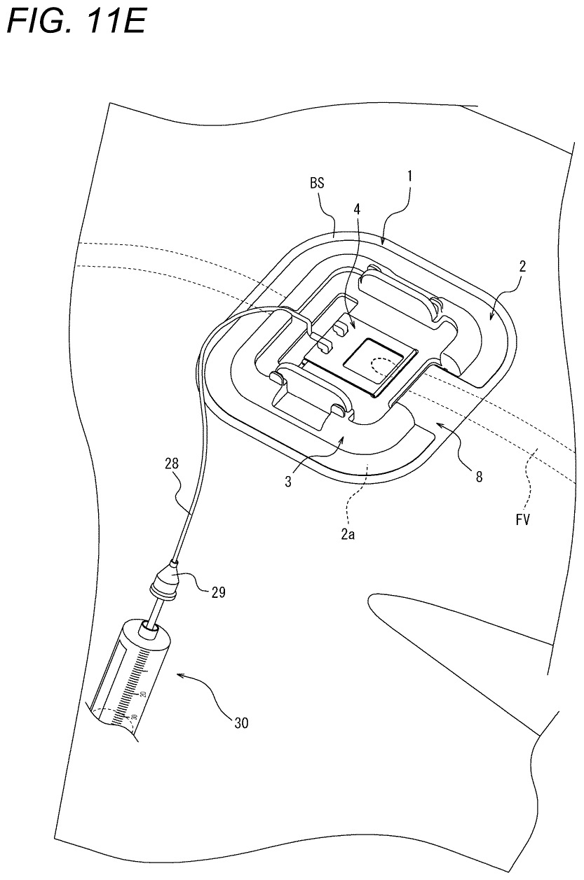

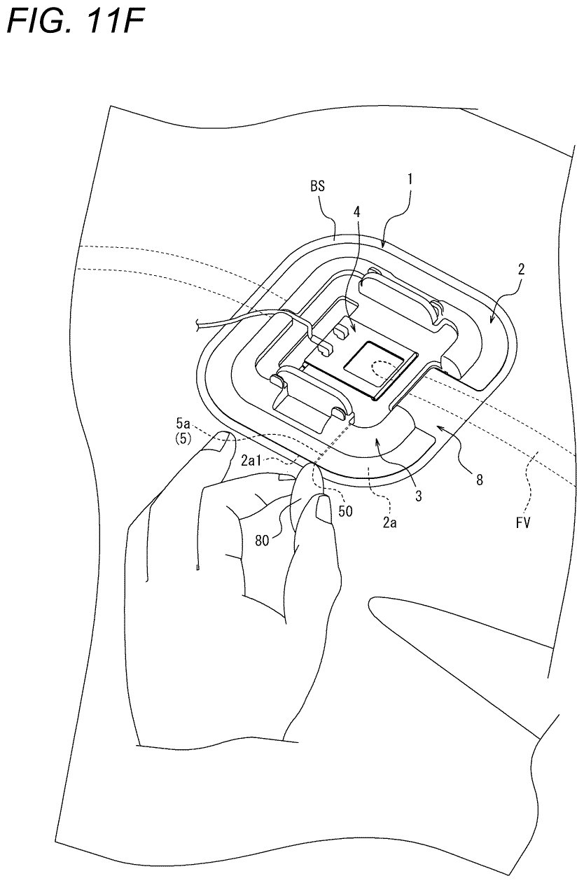

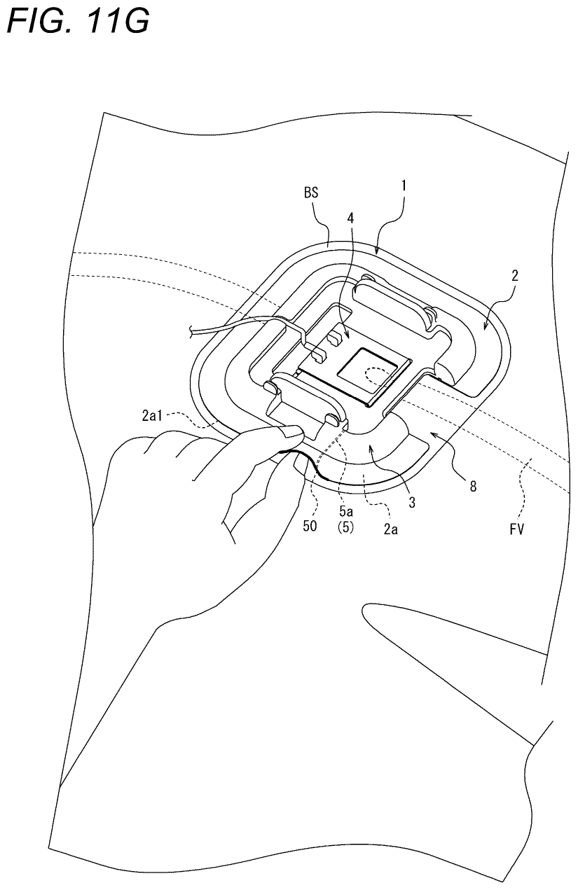

Next, a living body compression method performed on the living body surface using the compression device 1 according to the present disclosure is described. FIG. 10 is a flowchart illustrating an example of the living body compression method performed on the living body surface using the compression device 1. The living body compression method illustrated in FIG. 10 includes an example of a method for detaching the compression device 1 according to the present disclosure. The living body compression method illustrated in FIG. 10 includes an adhering step S1, a first compressing step S2, a removing step S3, a second compressing step S4, an impregnating step S5, and a detaching step S6. FIGS. 11A and 11B are views illustrating an outline of the adhering step S1. FIG. 11C is a view illustrating an outline of the first compressing step S2. FIG. 11D is a view illustrating an outline of the removing step S3. FIG. 11E is a view illustrating an outline of the second compressing step S4. FIG. 11F is a view illustrating an outline of the impregnating step S5. FIG. 11G is a view illustrating an outline of the detaching step S6.

The living body compression method illustrated in FIGS. 10, 11A to 11G is the living body compression method for compressing a living body surface BS to narrow or occlude a perforation leading from the living body surface to the vein without occluding the vein. The perforation is formed by removing a sheath as an insertion member for medical use 100 in a state inserted from the living body surface BS into the vein such as the femoral vein, for example, through connective tissue. By the living body compression method herein illustrated, hemostasis after removal of the sheath as the insertion member for medical use 100 can be performed. First, the perforation formed after removal of the insertion member for medical use 100 is described with reference to FIGS. 21A and 21B. FIG. 21A illustrates a state in which the sheaths as the insertion members for medical use 100 are inserted from the living body surface BS into a femoral vein FV through connective tissue CT. Although FIG. 21A illustrates three sheaths as the insertion members for medical use 100, the number of sheaths may be two or less or four or more. FIG. 21B illustrates a state after removal of the sheaths as the insertion members for medical use 100 from the state illustrated in FIG. 21A. As illustrated in FIG. 21B, the perforations P are formed between the living body surface BS and the femoral vein FV by removing the sheaths as the insertion members for medical use 100. In the living body compression method illustrated in FIGS. 10, and 11A to 11G, the perforation P can be narrowed or occluded without occluding the femoral vein FV. Therefore, even in a case of hemostasis of bleeding from the vein at a deep position from the living body surface, it is not necessary to narrow or occlude the vein itself, and hemostasis can be performed more efficiently. Hereinafter, steps S1 to S6 are described in detail with reference to FIGS. 11A to 11G.

FIG. 11A illustrates a state in which the sheath as the insertion member for medical use 100 is inserted from the living body surface BS into the femoral vein FV (refer to FIGS. 21A and 21B). FIG. 11B illustrates a state in which the attachment of the compression device 1 to a predetermined position on the living body surface BS is completed in a state in which the sheath as the insertion member for medical use 100 is inserted into the living body. FIG. 11A illustrates a state before use in which the adhesive surface 2a is covered with a liner 70. In contrast, FIG. 11B illustrates a use state after the liner 70 is peeled off from the adhesive surface 2a.

As illustrated in FIGS. 11A and 11B, the compression device 1 is attached to the living body surface by adhering the adhesive surface 2a of the adhesive sheet 2 to the living body surface. Specifically, as illustrated in FIG. 11B, the adhesive surface 2a is adhered to the living body surface BS in a state in which a portion exposed to the outside from the living body surface BS of the sheath as the insertion member for medical use 100 inserted into the living body from the living body surface BS is received by the receiving portion 8.

Next, as illustrated in FIG. 11C, a syringe 30 as a fluid supply instrument is connected to the connection portion 29 of the tube 28. Air is supplied to the housing space 4a1 (refer to FIG. 7) of the expansion portion 4a (refer to FIG. 7 and the like) of the compression device 1 through the tube 28 to expand the expansion portion 4a. By doing so, it is possible to compress in advance the vicinity of the wound on the living body surface BS before removing the sheath as the insertion member for medical use 100 from the living body surface BS. In other words, compression of the living body surface BS is started in a state in which the sheath as the insertion member for medical use 100 is inserted from the living body surface BS into the femoral vein FV as the vein through the connective tissue CT (refer to FIGS. 21A and 21B). In this manner, compression is performed before the insertion member for medical use 100 is removed from the living body surface BS. As a result, the living body surface BS can be compressed immediately after the removal of the sheath as the insertion member for medical use 100. Therefore, the perforation P (refer to FIG. 21B) extending from the living body surface BS to the femoral vein FV (refer to FIGS. 21A and 21B) can be narrowed or occluded immediately after removal of the sheath.

Next, as illustrated in FIG. 11D, the sheath as the insertion member for medical use 100 is removed from the living body surface BS. By the removal of the sheath, the perforation P illustrated in FIG. 21B is formed. If the living body surface BS is not compressed at all in this state, bleeding occurs from the femoral vein FV out of the living body through the perforation P and the wound on the living body surface BS. However, in the living body compression method herein illustrated, as illustrated in FIG. 11C, the living body surface BS is compressed in advance before the sheath as the insertion member for medical use 100 is removed from the living body surface BS. Therefore, the living body surface BS can be compressed immediately after the removal of the sheath so that the perforation P (refer to FIG. 21B) is narrowed or occluded, and an amount of bleeding immediately after the removal of the sheath can be suppressed.

Next, as illustrated in FIG. 11E, the syringe 30 as the fluid supply instrument is connected again to the connection portion 29 of the tube 28. Through the tube 28, air is supplied again or removed to pressurize or depressurize the housing space 4a1 (refer to FIG. 7) of the expansion portion 4a (refer to FIG. 7 and the like) of the compression device 1. In other words, the compressing force on the living body surface BS is adjusted after removal of the sheath as the insertion member for medical use 100. As a result, it is possible to significantly reduce the amount of bleeding or stop the bleeding by further narrowing or occluding the perforation P (refer to FIG. 21B) without occluding the femoral vein FV (refer to FIGS. 21A and 21B) by adjusting the compressing force on the living body surface BS.

More specifically, in a case where bleeding is confirmed after the removal of the sheath, the compressing force is slowly increased to pressurize until hemostasis is achieved. In contrast, in a case where hemostasis is confirmed after removal of the sheath, the compressing force is slowly reduced to depressurize until bleeding is confirmed. After bleeding is confirmed, the compressing force is slowly increased to pressurize until hemostasis is achieved. By doing so, it is possible to suppress the occlusion of the femoral vein FV (refer to FIGS. 21A and 21B) due to excessive pressurization.

Hemostasis can be completed by maintaining a compression state for several hours (for example, two hours to six hours).

Before the adhesive surface 2a of the adhesive sheet 2 is peeled off from the living body surface BS, as illustrated in FIG. 11F, the release liquid is allowed to permeate into the liquid guide member 5. The release liquid can be allowed to permeate into the liquid guide member 5 from the liquid introduction portion 50 of the liquid guide member 5 provided at the position of the outer edge of the adhesive sheet 2 corresponding to the outer edge 2a1 of the adhesive surface 2a, for example, using a liquid supply member 80 such as absorbent cotton impregnated with the release liquid. Then, the liquid guide member 5 is impregnated with the release liquid. The release liquid spreads into a minute gap between the adhesive surface 2a and the living body surface BS via the liquid guide member 5. By doing so, the adhering force of the adhesive surface 2a to the living body surface BS is weakened, and the adhesive surface 2a is easily peeled off from the living body surface BS.

Next, as illustrated in FIG. 11G, after the liquid guide member 5 is impregnated with the release liquid, the adhesive surface 2a of the adhesive sheet 2 is peeled off from the living body surface BS, so that the compression device 1 is detached from the living body surface BS. At that time, it is preferable to peel off the adhesive surface 2a starting from the position on the adhesive surface 2a at which the liquid guide member 5 is adhered. In the vicinity of the liquid guide member 5, the release liquid easily spreads between the adhesive surface 2a and the living body surface BS. Therefore, a portion that serves as a starting point of a peeling operation is easily formed, the portion separated from the living body surface BS and can be gripped with fingers, on the outer edge of the adhesive sheet 2. As illustrated in FIG. 11G, a portion separated from the living body surface BS out of the outer edge of the adhesive sheet 2 can be pinched with the fingers to peel off the adhesive surface 2a from the living body surface BS. By doing so, the adhesive surface 2a of the adhesive sheet 2 is peeled off from the living body surface BS to detach the compression device 1 from the living body surface BS.

As described above, in the living body compression method herein illustrated, the perforation P (refer to FIG. 21B) is narrowed or occluded without occluding the femoral vein FV (refer to FIGS. 21A and 21B). In a case of hemostasis of the vein, hemostasis can be performed by narrowing or occluding the perforation P (refer to FIG. 21B). In contrast, for example, in case of hemostasis of the femoral artery, even when only the perforation is occluded, hemostasis cannot be achieved since blood leaks and spreads into the connective tissue CT (refer to FIGS. 21A and 21B). In a case of hemostasis of the femoral artery, it is necessary to apply a large-scale measure such as a method for strongly compressing the artery itself so as to narrow or occlude the same and a method for blocking a hole of the artery wall.

Therefore, in the above-described living body compression method, the living body surface BS is preferably compressed to a position at which a compression depth from the living body surface BS is 5 mm to 20 mm. By setting the compression depth within the above-described range, the compression state in which the perforation P (refer to FIG. 21B) is narrowed or occluded is easily implemented without occluding the vein. The compression depth is more preferably 5 mm to 15 mm, and still more preferably 8 mm to 12 mm.

Moreover, in the above-described living body compression method, the living body surface BS is preferably compressed at 10 g/cm² to 600 g/cm² from the living body surface BS. The compression pressure is a pressure after removal of the sheath as the insertion member for medical use 100, and does not mean the above-described compressing force before removal of the sheath. By setting the compression pressure within the above-described range, the compression state in which the perforation P (refer to FIG. 21B) is narrowed or occluded is easily implemented without occluding the vein. The compression pressure is more preferably 50 g/cm² to 400 g/cm², and still more preferably 100 g/cm² to 300 g/cm².

The living body surface BS is preferably compressed in a direction orthogonal to an extending direction of the perforation P (refer to FIG. 21B). The phrase of “compressed in a direction orthogonal to an extending direction of the perforation” is not limited to compressing only in the direction orthogonal to the extending direction of the perforation, but also includes compressing in a direction inclined at an angle equal to or less than a predetermined angle (for example, 30 degrees or less) with respect to the direction orthogonal to the extending direction of the perforation. The compression device 1 of the present embodiment can compress the living body surface BS in the direction orthogonal to the extending direction of the perforation P (refer to FIG. 21B).

Specifically, the expansion portion as the expansion portion 4a of the present embodiment can be expanded in the direction inclined with respect to the thickness direction A as described above. By doing so, the living body surface can be compressed in the direction orthogonal to the extending direction of the perforation P (refer to FIG. 21B). Specifically, as illustrated in FIGS. 21A and 21B, the sheath as the insertion member for medical use 100 is inserted not in the direction (the same direction as the thickness direction A) orthogonal to the living body surface BS but in a direction inclined to one side with respect to the direction orthogonal to the living body surface BS. Therefore, as illustrated in FIG. 21B, the extending direction of the perforation P is also inclined with respect to the direction orthogonal to the living body surface BS. Therefore, with the expansion portion 4a expandable in a direction inclined to a side opposite to the extending direction of the perforation P with respect to the thickness direction A that is the direction orthogonal to the living body surface BS (hereinafter, sometimes referred to as an “inclined direction F”), the living body surface BS is easily compressed in the direction orthogonal to the extending direction of the perforation P. Therefore, it is easy to implement the compression device 1 that narrows or occludes the perforation P without occluding the vein such as the femoral vein FV in FIGS. 21A and 21B. FIG. 22 is a view illustrating a state in which the perforation P illustrated in FIG. 21B is narrowed or occluded by the compression device 1. As illustrated in FIG. 22, according to the compression device 1, the perforation P is more easily narrowed or occluded without further occluding the vein such as the femoral vein FV.

According to the living body compression method illustrated in FIGS. 10, and 11A to 11G, hemostasis can be performed by narrowing or occluding the perforation P (refer to FIG. 21B) without occluding the vein such as the femoral vein FV. In particular, by implementing the above-described living body compression method with the compression device 1, it is possible to perform hemostasis by a simple method without the need of compression by a medical worker's hand, use of a large-scale hemostatic instrument or the like.

### Compression on Living Body Surface by Compression Device 1

As illustrated in FIG. 22, in the compression device 1, in a state in which the adhesive sheet 2 is mounted on the living body, the expansion portion 4a can compress the living body surface in the inclined direction F inclined with respect to a perpendicular direction (the same direction as the thickness direction A in FIG. 22, and is the vertical direction in FIG. 22; hereinafter, simply referred to as the “perpendicular direction”) perpendicular to the living body surface BS. By doing so, as illustrated in FIG. 22, the perforation P is easily narrowed or occluded without occluding the vein such as the femoral vein FV.

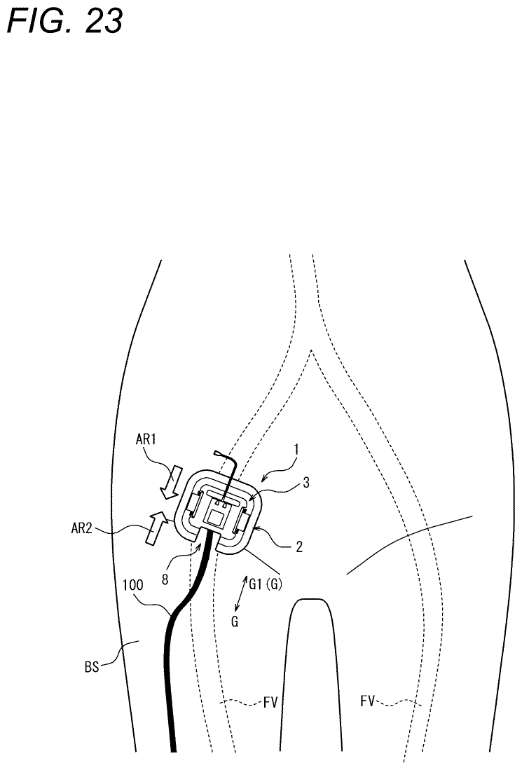

FIG. 23 is a front view of the state illustrated in FIG. 22 as seen from the living body surface BS side. In other words, FIG. 23 illustrates a front view of the living body surface BS at a position compressed by the compression device 1. Herein, the phrase “a front view of the living body surface at a position compressed by the compression device” means a state in which a part of the living body surface to be compressed by the compression device is seen in a direction perpendicular to the part before compression. FIG. 23 illustrates a front view of the groin. In the front view illustrated in FIG. 23, a direction (refer to outlined arrow “AR1” in FIG. 23) in which the living body surface BS is compressed is opposed to a sheath insertion direction G1 (refer to outlined arrow “AR2” in FIG. 23) from the living body surface BS toward the vein in the extending direction G of the perforation P. That is, the direction in which the compression device 1 compresses the living body surface BS is opposed to the sheath insertion direction G1 in the front view illustrated in FIG. 23. By doing so, the perforation P (refer to FIG. 21B and FIG. 22) is easily narrowed or occluded without occluding the vein such as the femoral vein FV.

In other words, as illustrated in FIG. 22, the extending direction G of the perforation P is inclined with respect to the living body surface BS and is also inclined with respect to the perpendicular direction (the vertical direction in FIG. 22) perpendicular to the living body surface BS. As illustrated in FIG. 22, the direction in which the living body surface BS is compressed by the compression device 1 is inclined with respect to the living body surface BS, and is also inclined with respect to the perpendicular direction (the vertical direction in FIG. 22) perpendicular to the living body surface BS. Moreover, as illustrated in FIG. 22, the extending direction G of the perforation P is inclined to the opposite side to the inclined direction F as the direction in which the living body surface is compressed by the compression device 1 with respect to the perpendicular direction (the vertical direction in FIG. 22). That is, the living body surface is compressed by the compression device 1 so that the compression direction intersects with the extending direction G of the perforation P. As a result, the perforation P can be efficiently narrowed or occluded.

### Second Embodiment

Next, a compression device 101 as a second embodiment of the present disclosure is described with reference to FIG. 12. FIG. 12 is a bottom view of the compression device 101. The compression device 101 is different from the above-described compression device 1 (refer to FIG. 1 and the like) only in arrangement position of a liquid guide member 105, and other configurations are the same. Therefore, only the difference is herein described, and description of the common configuration is omitted.

As illustrated in FIG. 12, the liquid guide member 105 of the present embodiment protrudes outward from an outer edge 2a1 of an adhesive surface 2a. The liquid guide member 105 of the present embodiment protrudes inward from an inner edge 2a2 of the adhesive surface 2a.

More specifically, the liquid guide member 105 of the present embodiment is provided with an outer extended portion 105b and an inner extended portion 105c in addition to a main body 5a adhered in the adhesive surface 2a of an adhesive sheet 2.

The outer extended portion 105b is continuous to the main body 5a and extends outward from the outer edge 2a1 of the adhesive surface 2a. More specifically, the outer extended portion 105b of the present embodiment protrudes outward from an outer edge of the adhesive sheet 2 in plan view (refer to FIG. 12) as seen in a thickness direction A. That is, the outer extended portion 105b of the present embodiment is provided outside the adhesive sheet 2 at a position not overlapping the adhesive sheet 2 in plan view (refer to FIG. 12) as seen in the thickness direction A.

By providing such outer extended portion 105b, a release liquid is easily allowed to permeate into the liquid guide member 105. That is, when the release liquid is allowed to permeate into the liquid guide member 105, it is not necessary to attach the release liquid to an end of the main body 5a covered with the adhesive surface 2a, and the release liquid can be attached to the outer extended portion 105b not covered with the adhesive surface 2a. That is, the outer extended portion 105b can be used as a liquid introduction portion 50 of the release liquid.

Moreover, by providing the outer extended portion 105b, a medical worker can grip the outer extended portion 105b and execute a peeling operation when peeling the adhesive surface 2a off from a living body surface. That is, the outer extended portion 105b can be used as an operation portion when the adhesive surface 2a of the adhesive sheet 2 is peeled off from the living body surface. The medical worker moves the outer extended portion 105b in an upward direction A2 so as to separate the same from the living body surface while gripping the outer extended portion 105b. As a result, the adhesive surface 2a can be peeled off from the living body surface from a position in the vicinity of a portion to which the liquid guide member 105 is adhered of the adhesive surface 2a.

The inner extended portion 105c is continuous to the main body 5a and extends inward from the inner edge 2a2 of the adhesive surface 2a. More specifically, the inner extended portion 105c of the present embodiment protrudes inward from the inner edge of the adhesive sheet 2 in plan view (refer to FIG. 12) as seen in the thickness direction A. That is, the inner extended portion 105c of the present embodiment is provided inside the adhesive sheet 2 at a position not overlapping the adhesive sheet 2 in plan view (refer to FIG. 12) as seen in the thickness direction A. In this manner, the liquid guide member 105 may be provided with the inner extended portion 105c. Note that, it is preferable that the liquid guide member 105 is not provided with the inner extended portion 105c as in the first embodiment described above. When there is the inner extended portion 105c, the release liquid that permeates into the main body 5a from the outer extended portion 105b easily leaks out of the adhesive surface 2a through the inner extended portion 105c. Since the liquid guide member 105 is not provided with the inner extended portion 105c, it is possible to suppress the above-described release liquid from leaking out of the adhesive surface 2a. As a result, the release liquid easily spreads between the adhesive surface 2a and the living body surface, and peelability of the adhesive surface 2a of the adhesive sheet 2 can be improved.

The arrangement position of the liquid guide member 105 in a direction along the outer edge of the adhesive sheet 2 is different from the arrangement position of the liquid guide member 5 of the first embodiment described above in the same direction, but this position is not particularly limited. The liquid guide member 105 may be arranged at any other position in the direction along the outer edge of the adhesive sheet 2, for example, at the position similar to that of the liquid guide member 5 of the first embodiment. The compression device 101 of the present embodiment is provided with only one liquid guide member 105, but may be provided with a plurality of liquid guide members 105 arranged at different positions in the direction along the outer edge of the adhesive sheet 2.

### Third Embodiment

Next, a compression device 201 as a third embodiment of the present disclosure is described with reference to FIG. 13. FIG. 13 is a bottom view of the compression device 201. The compression device 201 is different from the above-described compression device 1 (refer to FIG. 1 and the like) only in arrangement position of a liquid guide member 205, and other configurations are the same. Therefore, only the difference is herein described, and description of the common configuration is omitted.

As illustrated in FIG. 13, the liquid guide member 205 of the present embodiment protrudes outward from an outer edge 2a1 of an adhesive surface 2a. The liquid guide member 205 of the present embodiment does not protrude inward from an inner edge 2a2 of the adhesive surface 2a.

More specifically, the liquid guide member 205 of the present embodiment is provided with an outer extended portion 105b in addition to a main body 205a adhered in the adhesive surface 2a of an adhesive sheet 2.

The configuration of the outer extended portion 105b is similar to that of the second embodiment described above. Therefore, the description of the outer extended portion 105b is herein omitted.

The main body 205a of the present embodiment does not extend to the position of the inner edge 2a2 of the adhesive surface 2a, and terminates in the adhesive surface 2a. That is, the liquid guide member 205 of the present embodiment is not provided with the inner extended portion 105c (refer to FIG. 12) as illustrated in the second embodiment described above, and the main body 205a terminates in the adhesive surface 2a before reaching the position of the inner edge 2a2 of the adhesive surface 2a. By doing so, it becomes more difficult for a release liquid that permeates into the main body 205a from the outer extended portion 105b to leak from the position of the inner edge 2a2 of the adhesive surface 2a. As a result, the release liquid that permeates from the outer extended portion 105b into the main body 205a easily spreads between the adhesive surface 2a and the living body surface, and peelability of the adhesive surface 2a of the adhesive sheet 2 can be further improved.

The arrangement position of the liquid guide member 205 in a direction along the outer edge of the adhesive sheet 2 is different from the arrangement position of the liquid guide member 5 of the first embodiment described above in the same direction, but this position is not particularly limited. The liquid guide member 205 may be arranged at any other position in the direction along the outer edge of the adhesive sheet 2, for example, at the position similar to that of the liquid guide member 5 of the first embodiment. The compression device 201 of the present embodiment is provided with only one liquid guide member 205, but may be provided with a plurality of liquid guide members 205 arranged at different positions in the direction along the outer edge of the adhesive sheet 2.

### Fourth Embodiment

Next, a compression device 301 as a fourth embodiment of the present disclosure is described with reference to FIG. 14. FIG. 14 is a bottom view of the compression device 301. The compression device 301 is different from the above-described compression device 1 (refer to FIG. 1 and the like) only in arrangement position of a liquid guide member 305 and a shape of the liquid guide member 305, and other configurations are the same. Therefore, only the difference is herein described, and description of the common configuration is omitted.

As illustrated in FIG. 14, the liquid guide member 305 of the present embodiment protrudes outward from an outer edge 2a1 of an adhesive surface 2a. The liquid guide member 305 of the present embodiment does not protrude inward from an inner edge 2a2 of the adhesive surface 2a.

More specifically, the liquid guide member 305 of the present embodiment is provided with an outer extended portion 305b in addition to a main body 305a adhered in the adhesive surface 2a of an adhesive sheet 2.

The outer extended portion 305b of the present embodiment is different from the outer extended portion 105b of the second embodiment described above only in outer shape, and has a substantially similar function. Therefore, the description of outer extended portion 305b is herein omitted.

The main body 305a of the present embodiment does not extend to the position of the inner edge 2a2 of the adhesive surface 2a, and terminates in the adhesive surface 2a. That is, the liquid guide member 305 of the present embodiment is not provided with the inner extended portion 105c (refer to FIG. 12) as illustrated in the second embodiment described above, and the main body 305a terminates in the adhesive surface 2a before reaching the position of the inner edge 2a2 of the adhesive surface 2a. By doing so, it becomes more difficult for a release liquid that permeates into the main body 305a from the outer extended portion 305b to leak from the position of the inner edge 2a2 of the adhesive surface 2a. As a result, the release liquid that permeates from the outer extended portion 305b into the main body 305a easily spreads between the adhesive surface 2a and the living body surface, and peelability of the adhesive surface 2a of the adhesive sheet 2 can be further improved.

Moreover, the main body 305a of the present embodiment is provided only at a position outside a base member 3 in plan view (refer to FIG. 14) as seen in a thickness direction A.

More specifically, the adhesive sheet 2 of the present embodiment is provided with a fixing portion 41 and a non-fixing portion 42. The fixing portion 41 is a portion of the adhesive sheet 2 to which the base member 3 is fixed. The non-fixing portion 42 is a portion of the adhesive sheet 2, the portion located outside the fixing portion 41 in plan view (refer to FIG. 14) as seen in the thickness direction A to which the base member 3 is not fixed. In other words, the fixing portion 41 of the present embodiment is a portion of the adhesive sheet 2, the portion overlapping with a frame portion 3a, which is an outer edge portion of the base member 3, in plan view (refer to FIG. 14) as seen in the thickness direction A. The non-fixing portion 42 of the present embodiment is a portion of the adhesive sheet 2, the portion not overlapping with the base member 3 outside the outer edge of the base member 3 in plan view (refer to FIG. 14) as seen in the thickness direction A.

The adhesive surface 2a of the adhesive sheet 2 of the present embodiment is provided with a first adhesive portion 43 and a second adhesive portion 44. The first adhesive portion 43 is a portion provided at the position of the fixing portion 41 out of the adhesive surface 2a. The second adhesive portion 44 is a portion of the adhesive surface 2a including the outer edge 2a1 of the adhesive surface 2a and provided at the position of the non-fixing portion 42.

The main body 305a of the liquid guide member 305 of the present embodiment is adhered only to the second adhesive portion 44 in plan view (refer to FIG. 14) as seen in a thickness direction A. That is, the main body 305a of the liquid guide member 305 of the present embodiment is not adhered to the first adhesive portion 43 provided at the position of the fixing portion 41 of the adhesive sheet 2. In short, the first adhesive portion 43 provided at the position of the fixing portion 41 is a portion immediately below the position at which the base member 3 is fixed to the adhesive sheet 2. While the compression device 301 compresses the living body surface, a large force in an upward direction A2 to separate from the living body surface is likely to act on the first adhesive portion 43 by an expansion portion 4a. That is, the first adhesive portion 43 is more easily peeled off than the second adhesive portion 44. Therefore, the main body 305a of the liquid guide member 305 is not adhered to the first adhesive portion 43, and an adhering area of the first adhesive portion 43 to the living body surface is secured. As a result, it is possible to suppress peeling of the first adhesive portion 43 from the living body surface while the compression device 301 compresses the living body surface.

Next, the shape of the liquid guide member 305 is described. The liquid guide member 5 (refer to FIGS. 8, 9 and the like) of the first embodiment is formed of the linear member, but the liquid guide member 305 of the present embodiment is formed of a belt-shaped member. With such a shape, an impregnation amount of the release liquid can be increased as compared with the above-described linear member. Moreover, an area of the outer extended portion 305b in plan view (refer to FIG. 14) as seen in the thickness direction A increases. Therefore, it is easy to attach the release liquid to the outer extended portion 305b. That is, the outer extended portion 305b can be more easily used as a liquid introduction portion 50. Moreover, the outer extended portion 305b is easily gripped by a medical worker. That is, the outer extended portion 305b can be more easily used as an operation portion.

The liquid guide member 305 of the present embodiment has a rectangular shape having a uniform width in plan view (refer to FIG. 14) as seen in the thickness direction A, but is not limited to this configuration. For example, the liquid guide member 305 may have a shape that does not have a uniform width provided with a wide portion or a narrow portion in plan view as seen in the thickness direction A.

The liquid guide member 305 of the present embodiment is arranged in the vicinity of a receiving portion 8, but is not limited to this position. That is, the arrangement position of the liquid guide member 305 in the direction along the outer edge of the adhesive sheet 2 is not particularly limited. The liquid guide member 305 may be arranged at any other position in the direction along the outer edge of the adhesive sheet 2, for example, at the position similar to that of the liquid guide member 5 of the first embodiment. The compression device 301 of the present embodiment is provided with only one liquid guide member 305, but the compression device 301 may be provided with a plurality of liquid guide members 305 arranged at different positions in the direction along the outer edge of the adhesive sheet 2 as illustrated in FIG. 15.

Moreover, as illustrated in FIG. 16, the compression device 301 may be a liquid guide member 1305 extending in a substantially C-shape arranged in an entire region in the direction along the outer edge of the adhesive sheet 2.

### Fifth Embodiment

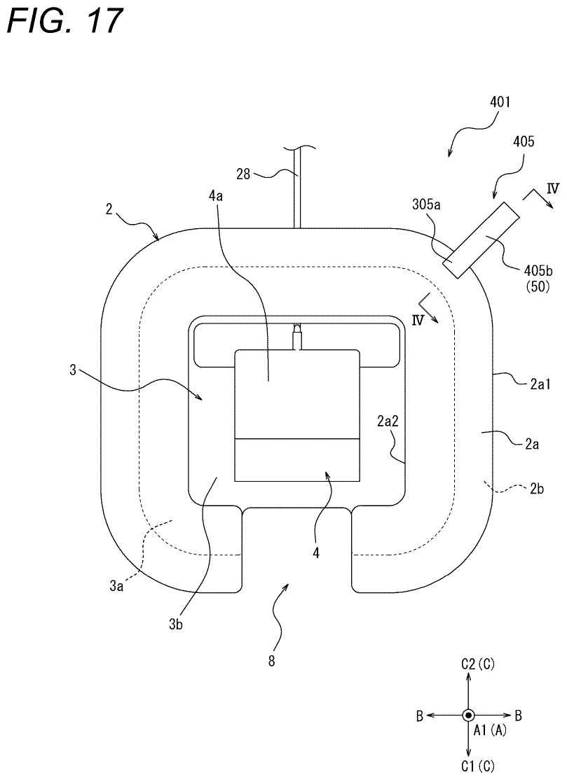

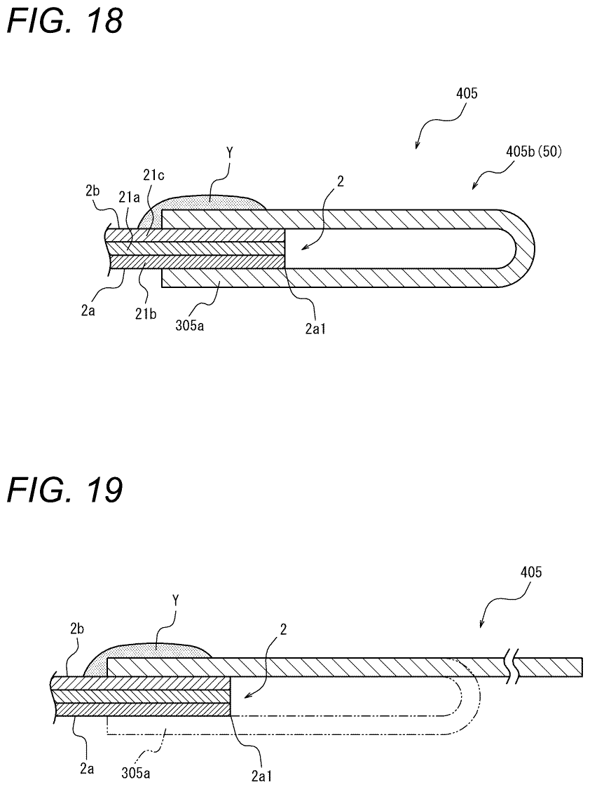

Next, a compression device 401 as a fifth embodiment of the present disclosure is described with reference to FIGS. 17 to 19 FIG. 17 is a bottom view of the compression device 401. FIG. 18 is a cross-sectional view taken along line IV-IV in FIG. 17. FIG. 19 is a cross-sectional view at the same position as FIG. 18, and is a view illustrating a state in which a main body 305a of a liquid guide member 405 is peeled off from an adhesive surface 2a. The compression device 401 is different from the above-described compression device 1 (refer to FIG. 1 and the like) only in arrangement position of the liquid guide member 405 and a configuration of the liquid guide member 305, and other configurations are the same. Therefore, only the difference is herein described, and description of the common configuration is omitted.

As illustrated in FIG. 17, the liquid guide member 405 of the present embodiment protrudes outward from an outer edge 2a1 of an adhesive surface 2a. The liquid guide member 405 of the present embodiment does not protrude inward from an inner edge 2a2 of the adhesive surface 2a.

More specifically, the liquid guide member 405 of the present embodiment is provided with an outer extended portion 405b in addition to a main body 305a adhered in the adhesive surface 2a of an adhesive sheet 2.

Since the main body 305a of the present embodiment is similar to that of the above-described fourth embodiment (refer to FIG. 14), the description of the main body 305a is herein omitted.

As illustrated in FIG. 18, the outer extended portion 405b of the present embodiment is folded back outside the outer edge 2a1 of the adhesive surface 2a and fixed to a fixing surface 2b. A mode of fixing the outer extended portion 405b to the fixing surface 2b is not particularly limited. The outer extended portion 405b may be fixed to the fixing surface 2b by, for example, various adhesives Y such as a UV curable adhesive.

By providing the outer extended portion 405b, a medical worker can grip the outer extended portion 405b and execute a peeling operation when peeling the adhesive surface 2a off from a living body surface. That is, the outer extended portion 405b can be used as an operation portion when the adhesive surface 2a of the adhesive sheet 2 is peeled off from the living body surface. At that time, the main body 305a might be peeled off from the adhesive surface 2a. In the present embodiment, since the outer extended portion 405b is fixed to the fixing surface 2b, as illustrated in FIG. 19, even if the main body 305a is peeled off from the adhesive surface 2a, integrality of the adhesive sheet 2 and the liquid guide member 405 is maintained. Therefore, even when the main body 305a is peeled off from the adhesive surface 2a, the liquid guide member 405 can be continuously used as an operation portion.

A cross-sectional outer shape of the liquid guide member 405 is not particularly limited. The cross-sectional outer shape of the liquid guide member 405 may be, for example, various cross-sectional shapes such as a rectangular shape, a circular shape, and an oval shape. Therefore, the outer shape of the liquid guide member 405 is not particularly limited, and may be a belt-shaped member as in the present embodiment, or may be another outer shape such as a linear member.

The arrangement position of the liquid guide member 405 in the direction along the outer edge of the adhesive sheet 2 is not particularly limited. The liquid guide member 405 may be arranged at any other position in the direction along the outer edge of the adhesive sheet 2, for example, at the position similar to that of the liquid guide member 5 of the first embodiment. The compression device 401 of the present embodiment is provided with only one liquid guide member 405, but may be provided with a plurality of liquid guide members 405 arranged at different positions in the direction along the outer edge of the adhesive sheet 2.

### Sixth Embodiment

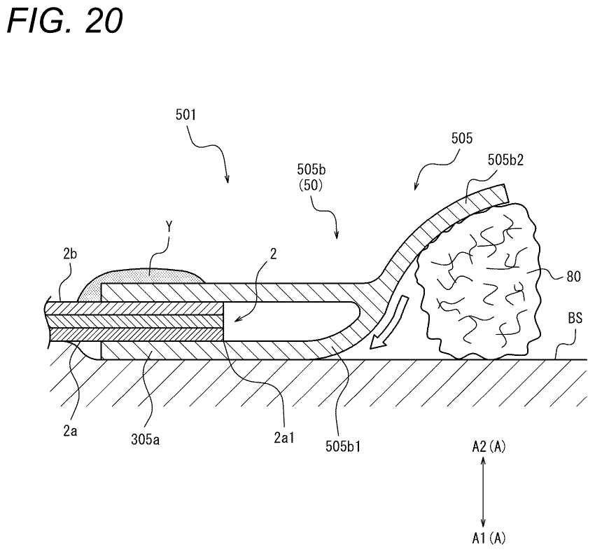

Next, a compression device 501 as a sixth embodiment of the present disclosure is described with reference to FIG. 20. FIG. 20 is a cross-sectional view in the vicinity of a liquid guide member 505 of the compression device 501. The compression device 501 is different from the above-described compression device 1 (refer to FIG. 1 and the like) only in configuration of the liquid guide member 505, and other configurations are the same. Therefore, only the difference is herein described, and description of the common configuration is omitted.

As illustrated in FIG. 20, the liquid guide member 505 of the present embodiment protrudes outward from an outer edge 2a1 of an adhesive surface 2a. The liquid guide member 505 of the present embodiment does not protrude inward from an inner edge 2a2 (not illustrated in FIG. 20, refer to FIG. 3 and the like) of the adhesive surface 2a.

More specifically, the liquid guide member 505 of the present embodiment is provided with an outer extended portion 505b in addition to a main body 305a adhered in the adhesive surface 2a of an adhesive sheet 2.

Since the main body 305a of the present embodiment is similar to that of the above-described fourth embodiment (refer to FIG. 14), the description of the main body 305a is herein omitted.

As illustrated in FIG. 20, the outer extended portion 505b of the present embodiment is folded back outside the outer edge 2a1 of the adhesive surface 2a and fixed to a fixing surface 2b. A mode of fixing the outer extended portion 505b to the fixing surface 2b is not particularly limited. The outer extended portion 505b may be fixed to the fixing surface 2b by, for example, various adhesives such as a UV curable adhesive.

By providing the outer extended portion 505b, a medical worker can grip the outer extended portion 505b and execute a peeling operation when peeling the adhesive surface 2a off from a living body surface. That is, the outer extended portion 505b can be used as an operation portion when the adhesive surface 2a of the adhesive sheet 2 is peeled off from the living body surface. At that time, the main body 305a might be peeled off from the adhesive surface 2a. In the present embodiment, since the outer extended portion 505b is fixed to the fixing surface 2b, even if the main body 305a is peeled off from the adhesive surface 2a, integrality of the adhesive sheet 2 and the liquid guide member 505 is maintained. Therefore, even when the main body 305a is peeled off from the adhesive surface 2a, the liquid guide member 505 can be continuously used as an operation portion.

At least a part of the outer extended portion 505b of the present embodiment is configured to be able to protrude in an upward direction A2 from the fixing surface 2b. More specifically, the outer extended portion 505b of the present embodiment is provided with an extension main portion 505b1 and a free end 505b2. One end of the extension main portion 505b1 is continuous to the main body 305a. The other end of the extension main portion 505b1 is fixed to the fixing surface 2b. The free end 505b2 protrudes from the extension main portion 505b1. In the outer extended portion 505b of the present embodiment, the free end 505b2 is configured to be able to protrude in the upward direction A2 from the fixing surface 2b. The free end 505b2 may be shaped in advance so as to protrude in the upward direction A2 from the fixing surface 2b, or may be flexible and flexibly deformable so as to protrude in the upward direction A2 from the fixing surface 2b.

As illustrated in FIG. 20, in a state in which the free end 505b2 is allowed to protrude in the upward direction A2, a peeling liquid is allowed to permeate into the free end 505b2 using a liquid supply member 80 such as absorbent cotton impregnated with the release liquid. At that time, a thickness direction A of the adhesive sheet 2 adhered to a living body surface BS of a patient lying on his/her back is a direction in a vertical direction. That is, since a downward direction A1 is a direction of gravity, the release liquid permeated into the free end 505b2 is likely to move in the downward direction A1 by a falling pressure (refer to hollow arrow in FIG. 20), and the permeation of the release liquid into the main body 305a can be promoted.

In the present embodiment, by providing the free end 505b2, the outer extended portion 505b that can protrude in the upward direction A2 from the fixing surface 2b is implemented, but this is not limited to this configuration. At least a part of the outer extended portion 505b is configured to be able to protrude in the upward direction A2 from the fixing surface 2b, another shape is also possible.

A cross-sectional outer shape of the liquid guide member 505 is not particularly limited. The cross-sectional outer shape of the liquid guide member 505 may be, for example, various cross-sectional shapes such as a rectangular shape, a circular shape, and an oval shape. Therefore, the outer shape of the liquid guide member 505 is not particularly limited, and may be a belt-shaped member as in the present embodiment, or may be another outer shape such as a linear member.

The arrangement position of the liquid guide member 505 in the direction along the outer edge of the adhesive sheet 2 is not particularly limited. The liquid guide member 505 may be arranged at any other position in the direction along the outer edge of the adhesive sheet 2, for example, at the position similar to that of the liquid guide member 5 of the first embodiment. The compression device 501 of the present embodiment is provided with only one liquid guide member 505, but may be provided with a plurality of liquid guide members 505 arranged at different positions in the direction along the outer edge of the adhesive sheet 2.

The compression device and the method for detaching the compression device according to the present disclosure are not limited to the specific configurations and steps illustrated in the above-described embodiments and variations, and various modifications, changes, and combinations are possible without departing from the scope of claims.

The detailed description above describes embodiments of a compression device and a method for detaching the compression device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A compression device comprising:
an adhesive sheet provided with an adhesive surface configured to be adhered to a living body surface;
an expansion member including an expandable expansion portion;
a base member fixed to a fixing surface on a side opposite to the adhesive surface of the adhesive sheet and configured to press the expansion portion in an expanded state toward the living body surface;
a liquid guide member into which a release liquid is configured to permeate, the liquid guide member provided with a main body adhered in the adhesive surface of the adhesive sheet; and
the main body extending to a position of an outer edge of the adhesive surface.

2. The compression device according to claim 1, wherein
the adhesive sheet is not arranged at a position overlapping the expansion portion but is located around the expansion portion in plan view as seen in a thickness direction of the adhesive sheet; and
the main body of the liquid guide member does not extend to a position of an inner edge of the adhesive surface but terminates in the adhesive surface.

3. The compression device according to claim 1, wherein
the adhesive sheet includes a fixing portion to which the base member is fixed, and a non-fixing portion located outside the fixing portion in plan view as seen in a thickness direction of the adhesive sheet to which the base member is not fixed;
the adhesive surface includes a first adhesive portion provided at a position of the fixing portion, and a second adhesive portion including the outer edge of the adhesive surface and is provided at a position of the non-fixing portion; and
wherein the main body of the liquid guide member is adhered only to the second adhesive portion.

4. The compression device according to claim 1, wherein the liquid guide member is provided with an outer extended portion continuous to the main body and extending outward from the outer edge of the adhesive surface.

5. The compression device according to claim 4, wherein the outer extended portion is folded back outside the outer edge of the adhesive surface and is fixed to the fixing surface.

6. The compression device according to claim 4, wherein
in a case where a direction from the adhesive surface toward the fixing surface in a thickness direction of the adhesive sheet is defined as an upward direction; and
at least a part of the outer extended portion is configured to be able to protrude in the upward direction from the fixing surface.

7. The compression device according to claim 1, further comprising:
the release liquid, the release liquid being a nonalcoholic silicone-based release agent or an alcohol.

8. The compression device according to claim 1, wherein the liquid guide member includes a plurality of voids into which the release liquid can permeate.

9. The compression device according to claim 8, wherein the liquid guide member is a nonwoven fabric, a woven fabric, a knitted fabric, a twisted yarn, or a porous body.

10. A compression device comprising:
an adhesive sheet provided with an adhesive surface configured to be adhered to a living body surface;
an expansion member including an expandable expansion portion;
a base member fixed to a fixing surface on a side opposite to the adhesive surface of the adhesive sheet and configured to press the expansion portion in an expanded state toward the living body surface; and
a liquid guide member into which a release liquid is configured to permeate, the liquid guide member provided with a main body adhered in the adhesive surface of the adhesive sheet, and wherein the main body of the guide member extends outward from an outer edge of the adhesive surface.

11. The compression device according to claim 10, wherein the main body of the guide member protrudes inward from an inner edge of the adhesive surface.

12. The compression device according to claim 10, wherein the adhesive sheet is not arranged at a position overlapping the expansion portion but is located around the expansion portion as seen in a thickness direction of the adhesive sheet.

13. The compression device according to claim 10, wherein the adhesive sheet includes:
a fixing portion to which the base member is fixed; and
a non-fixing portion located outside the fixing portion as seen in a thickness direction of the adhesive sheet to which the base member is not fixed.

14. The compression device according to claim 13, wherein the adhesive surface includes:
a first adhesive portion provided at a position of the fixing portion; and
a second adhesive portion including the outer edge of the adhesive surface and wherein the second adhesive portion is provided at a position of the non-fixing portion.

15. A method for detaching a compression device adhered to a living body surface, the compression device provided with an adhesive sheet provided with an adhesive surface that can be adhered to the living body surface, and a liquid guide member adhered in the adhesive surface into which a release liquid can permeate, the method comprising:
impregnating the liquid guide member with the release liquid by allowing the release liquid to permeate from a liquid introduction portion of the release liquid provided at a position of an outer edge of the adhesive surface or a position outside the outer edge of the adhesive surface in the liquid guide member; and
detaching the compression device from the living body surface by peeling the adhesive surface of the adhesive sheet off from the living body surface after the liquid guide member is impregnated with the release liquid.

16. The method according to claim 15, wherein the compression device further includes an expansion member including an expandable expansion portion, the adhesive sheet is not arranged at a position overlapping the expansion portion but is located around the expansion portion in plan view as seen in a thickness direction of the adhesive sheet, and the main body of the liquid guide member does not extend to a position of an inner edge of the adhesive surface but terminates in the adhesive surface, the method further comprises:
connecting a fluid supply instrument to a connector in communication with the expandable expansion portion; and
supplying a fluid via the connector in communication with expandable expansion portion to expand the expandable expansion portion to compress a vicinity of a wound on the living body surface.

17. The method according to claim 15, further comprising:
connecting the fluid supply instrument to the connector in communication with the expandable expansion portion; and
removing the fluid from the expandable expansion portion to deflate the expandable expansion portion.

18. The method according to claim 15, wherein the liquid guide member is provided with an outer extended portion extending outward from the outer edge of the adhesive surface.

19. The method according to claim 15, wherein the release liquid is a nonalcoholic silicone-based release agent or an alcohol.

20. The method according to claim 15, further comprising:
permeating the release liquid from a plurality of voids in the liquid guide member.
